(19) United States
(12) Patent Application Publication
(10) Pub. No.: US 2016/0067246 A1
(43) Pub. Date: Mar. 10, 2016

(54) USE OF PHOSPHODIESTERASE 5A INHIBITORS FOR THE TREATMENT OF MUSCULAR DYSTROPHY

(71) Applicant: CEDARS-SINAI MEDICAL CENTER, Los Angeles, CA (US)

(72) Inventors: Ronald G. Victor, Los Angeles, CA (US); Gail D. Thomas, Los Angeles, CA (US)

(73) Assignee: Cedars-Sinai Medical Center, Los Angeles, CA (US)

(21) Appl. No.: 14/782,883

(22) PCT Filed: Apr. 21, 2014
(86) PCT No.: PCT/US14/34806

§ 371 Date: Oct. 7, 2015

Related U.S. Application Data
(60) Provisional application No. 61/814,005, filed on Apr. 19, 2013.

(51) Int. Cl. A61K 31/4985; A61K 31/53; A61K 31/519

(52) U.S. Cl. CPC A61K 31/4985; A61K 31/519; A61K 31/53

(57) ABSTRACT

Described herein are methods of treating muscular dystrophy, including Becker's muscular dystrophy and Duchenne muscular dystrophy. The methods comprise administering a phosodiesterase 5A (PDE5A) inhibitor, such as tadalafil, to a subject in need thereof. Administering the PDE5A inhibitor has beneficial effects such as restoring functional sympatholysis, alleviating ischemic insult to dystrophin-deficient muscle membranes, reducing use-dependent muscle injury, and thus can slow muscular dystrophy disease progression.

### FIELD OF INVENTION

This invention relates to the treatment of muscular dystrophy.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Dystrophin is a large, rod-shaped, sarcolemmal protein that provides a physical link between the intracellular cytoskeleton and the extracellular matrix (7). With dystrophin deficiency, the sarcolemma is destabilized and the muscle fibers are susceptible to physical damage with repeated contraction (8). Dystrophin also is a scaffolding protein that targets other proteins to the sarcolemma. Among these is nNOSμ—a muscle-specific splice variant of the neuronal isoform of nitric oxide synthase (9,10)—which requires certain spectrin-like repeats in the mid-portion of dystrophin's rod domain and the adaptor protein α-syntrophin for sarcolemmal targeting (11). Dystrophin deficiency causes sarcolemmal nNOSμ deficiency: nNOSμ is reduced and the residual protein is misplaced from the sarcolemma to the cytosol (9-11).

With exercise of healthy skeletal muscle, sarcolemmal nNOSμ-derived nitric oxide (NO) attenuates local α-adrenergic vasoconstriction thereby optimizing perfusion to meet the metabolic demands of the active muscle (12-21). We previously found that this protective mechanism (termed functional sympatholysis) is lost in mdx mice (a model of BMD and DMD), nNOS null mice, and boys with DMD causing functional muscle ischemia (14,16). Repeated bouts of functional ischemia could accelerate use-dependent injury of muscle fibers already weakened by dystrophin deficiency (14,16,19).

Becker muscular dystrophy (BMD) is a progressive X-linked muscle wasting disease for which there is no treatment (1-3). Like the closely related disease Duchenne muscular dystrophy (DMD), BMD is caused by mutations in the gene encoding the cytoskeletal protein dystrophin. Whereas DMD is caused by out-of-frame mutations yielding no functional dystrophin, BMD is caused by in-frame mutations yielding truncated or reduced dystrophin protein (4,5). Despite a more protracted clinical course than DMD and an almost normal life span, BMD is a debilitating disease with progressive muscle weakness culminating in loss of ambulation; there also is an increased risk of heart failure due to an associated cardiomyopathy (3).

Duchenne muscular dystrophy (DMD) is a devastating X-linked muscle wasting disease for which there is no specific treatment. Affecting 1 in 3500 male births, DMD accounts for 80% of all cases of muscular dystrophy.^{1B} Dystrophic muscle undergoes slow progressive damage, leading to loss of ambulation at a very early age, with subsequent respiratory muscle weakness and cardiac failure. Despite our general understanding however, the exact pathophysiology of the disease remains largely unknown.

Although basic science on the dystrophinopathies has flourished, clinical translation has not (6). While much research and positive results have been seen in animal models, translation of those results to human subject has yet to be successful. This may be due to the fact that dosages given to animal models are typically hundreds or thousands times higher than what would be tolerable for human use. Thus, when lower dosages are given, the therapeutic effects seen in animal models are not present in human subjects. It has been known for a long time that the dystrophin gene is responsible causing muscular dystrophy and yet after all these years, treatment is limited to the use of corticosteroids, and cardioprotective medications to ease the effects of the disease, but does not treat or slow down the progression of the disease itself.

Thus, a therapeutic breakthrough is urgently needed for BMD and DMD.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of treating muscular dystrophy, comprising: providing a phosodiesterase 5A (PDE5A) inhibitor or a salt thereof and administering a quantity of a PDE5A inhibitor to a subject in need of treatment for muscular dystrophy.

In various embodiments, the muscular dystrophy treated can be Becker muscular dystrophy (BMD). In other embodiments, the muscular dystrophy treated can be Duchenne muscular dystrophy (DMD).

Various embodiments of the present invention provide for a method of restoring functional sympatholysis, comprising providing a phosodiesterase 5A (PDE5A) inhibitor or a salt thereof and administering a quantity of a PDE5A inhibitor to a subject in need of restoring functional sympatholysis.

Various embodiments of the present invention provide for a method of alleviating ischemic insult to dystrophin-deficient muscle membranes, comprising: providing a phosodiesterase 5A (PDE5A) inhibitor or a salt thereof and administering a quantity of a PDE5A inhibitor to a subject in need of alleviating ischemic insult to dystrophin-deficient muscle membranes.

Various embodiments of the present invention provide for a method of reducing use-dependent muscle injury, comprising: providing a phosodiesterase 5A (PDE5A) inhibitor or a salt thereof and administering a quantity of a PDE5A inhibitor to a subject in need of reducing use-dependent muscle injury.

Various embodiments of the present invention provide a method of alleviating post-exercise hyperemia, comprising: providing a phosodiesterase 5A (PDE5A) inhibitor or a salt thereof and administering a quantity of a PDE5A inhibitor to a subject in need of alleviating post-exercise hyperemia.

In various embodiments, the PDE5A inhibitor used in these methods can be tadalafil or a salt thereof. In various embodiments, the PDE5A inhibitor used in these methods can be sildenafil or a salt thereof. In various embodiments, the PDE5A inhibitor used in these methods can be vardenafil or a salt thereof.

In various embodiments, the subject can be human. In various embodiments, the subject can be an adult human. In various embodiments, the subject can be an adult, male human. In various embodiments, the subject can be a human child.

In various embodiments, the quantity of the PDE5A inhibitor used in these methods can be about 0.5 to 1.0 mg/kg body weight. In various embodiments, the quantity of the PDE5A inhibitor used in these methods can be administered prior to a period of heavy exertion. In various embodiments, the quantity of the PDE5A inhibitor used in these methods can be administered upon awakening. In various embodiments, the quantity of the PDE5A inhibitor used in these methods can be administered prior to a planned period of physical exertion. In various embodiments, the quantity of the PDE5A inhibitor used in these methods can be about 20 mg.

Various embodiments of the present invention provide for a method of treating Becker muscular dystrophy (BMD), comprising: providing about 20 mg of tadalafil or a salt thereof; and administering the about 20 mg of tadalafil or the salt thereof to an adult human subject in need of treatment for BMD. In various embodiments, the adult human subject can be male.

Various embodiments of the present invention provide for a method of treating Duchenne muscular dystrophy (DMD), comprising: providing about 0.5 mg/kg body weight of tadalafil, sildenafil or a salt thereof; and administering the about 0.5 mg/kg body weight of tadalafil, sildenafil or a salt thereof to an human subject in need of treatment for DMD. In various embodiments, the human subject can be a child.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-D show that functional sympatholysis is impaired in patients with Becker muscular dystrophy (BMD). (A) As shown in the representative trace from a healthy control subject, the lower body negative pressure (LBNP)-induced decrease in forearm muscle oxygenation (HbO₂+MbO₂) is greatly attenuated during mild handgrip exercise, demonstrating functional sympatholysis (i.e., normal exercise-induced attenuation of reflex vasoconstriction; grey oval). (B) The representative trace from a patient with BMD shows that sympatholysis is impaired, because handgrip fails to attenuate the LBNP response (red oval) indicating functional muscle ischemia. At the end of each experiment, an arm cuff was inflated to suprasystolic pressure to occlude the forearm circulation, producing a maximal decrease in muscle oxygenation to calculate total labile signal (TLS). (C, D) Summary data for 7 healthy controls (C) and 10 patients with BMD (D) expressed as a percentage of TLS (mean±SEM). *P<0.01 rest vs. exercise using a linear mixed-effects model.

FIG. 2A-D show that tadalafil alleviates functional muscle ischemia in a patient with Becker muscular dystrophy (BMD). (A) Tadalafil restores functional sympatholysis in a patient with BMD, as shown by the exercise-induced attenuation of the decrease in muscle oxygenation (HbO₂+MbO₂) with lower body negative pressure (LBNP) (grey oval). (B) In contrast, a placebo is without effect in the same patient, as shown by comparable LBNP responses during rest and exercise (red oval). (C, D) Summary data expressed as a percentage of total labile signal (TLS) (mean±SEM) from 10 patients with BMD all of whom completed the double-blind cross-over trial on tadalafil (C) and then placebo (D) or vice versa. *P<0.01, treatment condition×exercise condition interaction using a linear mixed-effects model.

FIG. 3A-B depict patient-specific data showing the consistency of the tadalafil effect. (A) With tadalafil, functional sympatholysis (exercise-induced attenuation of reflex vasoconstriction) is evident in nine of 10 patients with Becker muscular dystrophy (BMD), because the lower body negative pressure (LBNP)-induced decrease in forearm muscle oxygenation (HbO₂+MbO₂) is less during exercise (grey bars) than at rest (black bars). The exception was patient 10 (P10) in whom an adequate tadalafil blood concentration was achieved. (B) With placebo, the LBNP response is virtually identical during rest and exercise for each patient, except patient P9 in whom sympatholysis is clearly evident without tadalafil and possibly in patient P2 in whom sympatholysis is greatly augmented by tadalafil. Data are expressed as a percentage of totallabile signal (TLS) (mean±SEM).

FIG. 4 depicts immunohistochemistry of muscle biopsies from BMD patients. Muscle biopsy sections stained with haematoxylin and eosin (upper panel) from patient P5 (deletion of exons 45-48) and patient P9 (deletion of exons 14-44) show typical dystrophic hallmarks including variation in muscle fiber size, internal nuclei, fibrosis, and increase in fat tissue. Staining for dystrophin C-terminus (DYS-C) is present in muscle from both P5 and P9. Cytoplasmic staining for nNOS is evident in both P5 and P9 but not in control muscle. Sarcolemmal nNOS labeling is present in P9 but not detected in P5. Bottom panel shows higher magnification of fields enclosed in rectangles. Scale bars are 100 μm.

FIG. 5 depicts equivalence of functional sympatholysis in dominant vs. non-dominant arms. Two of 10 Becker muscular dystrophy (BMD) patients were left-handed; all other patients and all seven healthy controls were right-handed. In the cross-over trial, functional sympatholysis is impaired during placebo treatment because handgrip fails to attenuate the lower body negative pressure (LBNP)-induced response in forearm muscle oxygenation (HbO₂+MbO₂), indicating functional muscle ischemia. Tadalafil restores functional sympatholysis in BMD patients because the LBNP response is less during exercise. Sympatholysis is evident in healthy controls at baseline. In all cases, there are no differences between the dominant and non-dominant arms (P>0.05). Data are expressed as a percentage of total labile signal (TLS) (mean±SEM).*P<0.05, rest vs. exercise.

FIG. 6 depicts immunoblot experiments. Muscle samples from patient (P) 9, P5, and a healthy control subject (C) were probed with antibodies for the last 17 amino acids of the C-terminus of dystrophin (DYS-C), for amino acids 1181-1388 (encoding exons 26-32) of the dystrophin rod domain (DYS-Rod), and for desmin. P9 (deletion of exons 14-44) expresses DYS-C of reduced molecular weight but no DYS-rod due to the deletion of the epitope recognized by the antibody. P5 (deletion of exons 45-48) expresses both Dys-C and Dys-rod which are reduced both in molecular weight and amount. Desmin shows protein loading for the samples.

FIGS. 7A-C show that functional sympatholysis is impaired in patients with DMD and rescued by acute tadalafil treatment. (A) Representative tracing from a healthy control subject, showing that the LBNP-induced decrease in forearm muscle oxygenation (HbO₂+MbO₂) is greatly attenuated during mild handgrip exercise (grey circle), demonstrating functional sympatholysis. (B and C) Representative tracings from a patient with DMD, showing that sympatholysis is impaired because handgrip fails to attenuate the LBNP response (B, red circle), and that tadalafil rescues functional sympatholysis, shown by the exercise-induced attenuation of the decrease in muscle oxygenation with LBNP (C, black circle). At the end of each experiment, an arm cuff was inflated to suprasystolic pressure to occlude the forearm circulation, producing a maximal decrease in muscle oxygenation to calculate total labile signal (TLS).

FIG. 8 depicts summary data for 10 patients with DMD and 10 healthy controls showing that functional sympatholysis is impaired in patients with DMD and rescued by acute tadalafil treatment. Data expressed as a percentage of the total labile signal (TLS) (mean±SE). *P<0.01, rest versus exercise.

FIG. 9 shows that sildenafil equally restores functional sympatholysis in 6 patients with DMD. Data expressed as a percentage of the total labile signal (TLS) (mean±SE). *P<0.01, rest versus exercise.

FIG. 10 shows that post-exercise hyperemia is impaired in DMD and rescued by tadalafil. Data expressed as a percent change from rest to post-exercise (mean±SE). *P<0.05, compared to untreated. †P<0.05, patient vs. control.

FIG. 11 shows that post-exercise hyperemia is restored by sildenafil and tadalafil. Data expressed as a percentage change from rest to post-exercise (mean±SE). *P<0.05, compared to untreated.

FIGS. 12A-D depict twenty-four hour pharmacokinetic data in 9 boys with DMD, treated with two different phosphodiesterase (PDE) 5 inhibitors—sildenafil (A and B) and tadalafil (C and D)—with the dose starting at 0.5 mg/kg body weight, and escalating to 1.0 mg/kg body weight for each drug. To allow for adequate washout, each drug was separated by at least two-weeks. Data reported as mean±SE.

FIG. 13A depicts prophylactic cardiovascular treatment with either an angiotensin converting enzyme inhibitor (lisinopril, n=4) or an angiotensin receptor blocker (losartan, n=1) does not rescue functional sympatholysis in DMD, as demonstrated by a similar impairment exercise-induced attenuation of reflex vasoconstriction during LBNP compared to patients not undergoing prophylactic treatment (n=4). Data expressed as a percentage of the total labile signal (TLS) (mean±SE).

FIG. 13B depicts prophylactic cardiovascular treatment with either an angiotensin converting enzyme inhibitor (lisinopril, n=4) or an angiotensin receptor blocker (losartan, n=1) does not rescue functional sympatholysis in DMD, as demonstrated by a similar impairment exercise-induced attenuation of reflex vasoconstriction during LBNP compared to patients not undergoing prophylactic treatment (n=5). Data expressed as a percentage of the total labile signal (TLS) (mean±SE).

FIGS. 14A-D show functional sympatholysis is impaired in patients with DMD and rescued by acute tadalafil treatment. (A) Left panel shows a representative tracing from a healthy control subject, showing that the LBNP-induced decrease in forearm muscle oxygenation (HbO2+MbO2) is greatly attenuated during mild handgrip exercise (circle), demonstrating functional sympatholysis. At the end of each experiment, an arm cuff was inflated to supra-systolic pressure to occlude the forearm circulation, producing a maximal decrease in muscle oxygenation to calculate the total labile signal (TLS). Right panel shows summary forearm muscle oxygenation data from the 10 healthy control subjects studied at rest and during exercise expressed as a percentage of TLS. (B) Representative tracings from a patient with DMD at baseline (pre-treatment), showing that sympatholysis is impaired because handgrip fails to attenuate the LBNP response (circle). Summary data are shown in right panel for 10 boys with DMD. (C) Representative tracing from a DMD subject treated with Tadalafil, showing that the LBNP-induced decrease in forearm muscle oxygenation is greatly attenuated during mild handgrip exercise (circle). Summary data are shown in the right panel, for 10 patients with DMD. (D) Representative tracing from a DMD subject treated with Sildenafil, showing that the LBNP-induced decrease in forearm muscle oxygenation is greatly attenuated during mild handgrip exercise (circle). Summary data are shown in the right panel, for 6 patients with DMD. Data are reported as mean±SE.

FIGS. 15A-B show that sildenafil equally restores functional sympatholysis compared to tadalafil. (A) Lower-body negative pressure (LBNP)-induced decreases on forearm muscle oxygenation by near-infrared spectroscopy at rest, and during mild rhythmic handgrip exercise (n=6). Data expressed as a percentage of the total labile signal (TLS). Data reported as mean±SE. (B) Patient-specific data showing the effect of tadalafil and sildenafil on functional sympatholysis (exercise-induced attenuation of reflex vasoconstriction). P1-P6 received both sildenafil and tadalafil, whereas P7-P10 only received tadalafil. Severe movement artifact prevented data analysis in P9 during low dose tadalafil

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N. Y. 2001), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

“Treatment” and “treating,” as used herein refer to both therapeutic treatment and prophylactic or preventative measures, wherein the object is to prevent, slow down and/or lessen the disease even if the treatment is ultimately unsuccessful.

We show herein that the PDE5A inhibitor tadalafil rescues the abnormal vascular phenotype in the muscle of patients with BMD, fully restoring NO-dependent modulation of reflex vasoconstriction in exercising human skeletal muscle.

We also show herein that acute PDE5 inhibition alleviates functional muscle ischemia in boys with DMD. We also identified a brand-new vascular phenotype, that exercise-induced increases in post-exercise skeletal muscle blood flow is impaired in boys with DMD, and show that post-exercise hyperemia is also restored by acute PDE5 inhibition.

Treatment trials focused on BMD are scarce, as BMD is rarer than DMD (1 in 19,000 vs. 1 in 3,500 live male births, respectively) and clinically far more heterogeneous (3,11,33,34,36). Yet, BMD provides an elegant experiment of nature that in many patients eliminates sarcolemmal nNOSμ from birth, affords the opportunity to test a preemptive intervention in adult patients in an early stage of their disease, and provides insight into expected benefits from exon skipping which aims to convert DMD to BMD. Whereas most patients with DMD are treated with corticosteroids (deflazacort or prednisone) and many with prophylactic cardioprotective medication (1,2) that might affect sympatholysis, most of our BMD patients were taking no medication other than the study drug. We excluded BMD patients with heart failure or hypertension, which can impair sympatholysis via production of reactive oxygen species that destroy NO (19,20,37).

That functional sympatholysis was absent in nine of 10 adult ambulatory patients with BMD and normal LVEF—but was present in one patient shown to express sarcolemmal nNOSμ—provides new evidence in humans that sarcolemmal nNOSμ is essential for the normal modulation of sympathetic vasoconstriction in active skeletal muscle. In the absence of such modulation, the forearm muscles become ischemic when lightly exercised during mild orthostatic stress, simulating the common condition of a BMD patient performing repetitive arm activities of daily living while seated. The new data in adult patients with BMD extend our prior work showing that sympatholysis also is absent in pediatric patients with DMD (14) as well as in mdx mice (16), nNOS null mice (16), α-syntrophin null mice that lack sarcolemmal nNOSμ (19), and mdx transgenic mice expressing a dystrophin minigene which, like the common BMD mutations, produces a truncated dystrophin that cannot target nNOSμ to the sarcolemma (11).

A recent mouse study suggests that mdx mice have less sympathetic vasoconstriction to overcome during exercise due to impaired sympathetic neurotransmission, that is, a primary neural defect at the level of the sympathetic nerve terminals that is evident even in resting mdx skeletal muscle (38,39). Our data indicate that this finding in barbiturate-anesthetized mice is not applicable to conscious human patients with BMD, because the vasoconstrictor response to reflex sympathetic stimulation in resting BMD muscle is indistinguishable from normal. The reflex sympathetic vasoconstriction in resting DMD skeletal muscle also is not attenuated until the muscles are exercised (14).

A key finding of our study is that tadalafil alleviated microvascular ischemia and fully restored blood flow regulation in eight of nine BMD patients who lacked sympatholysis when given placebo. The tadalafil effect was both dramatic and immediate, occurring with a single dose. While not wishing to be bound by any particular theory, the inventors believe that PDE5A inhibitors boost a residual NO-cGMP signal arising from nNOSμ misplaced to the cytosol of dystrophin-deficient skeletal muscle. Cytosolic nNOS has been found in most patients with BMD (40) and indeed was detected in our two patients in whom muscle biopsy tissue was available for study. That tadalafil affected neither reflex vasoconstriction in resting BMD skeletal muscle nor systemic blood pressure suggested that the exercise-specific action of tadalafil in this setting does not involve either endothelial NOS (eNOS)-derived NO or central inhibition of sympathetic outflow.

Because tadalafil, unlike other PDE5A inhibitors, is specific for cGMP (without affecting cAMP at clinical doses) (41,42), our human data confirm but also extend prior mdx mouse studies implicating vascular NO-cGMP signaling as a potential new drug target for BMD and DMD. Asai et al. (28) showed that the normal increase in microvessel flow with electrically-evoked muscle contraction is blunted in anesthetized mdx mice and that dystrophic muscle histology can be improved by prenatal treatment with tadalafil; however, the authors did not report whether the vascular dysregulation also was improved by tadalafil. Kobayashi et al. (29) showed that single-dose tadalafil dramatically eliminates spasm of intramuscular arterioles (as well as indices of muscle injury and fatigue) in mdx mice after a brief bout of eccentric (i.e., injury-producing) exercise. Whereas the optimal dose in the mouse study of Kobayashi et al. was 500-1,000 times higher than the highest tadalafil doses used in clinical medicine, the 20 mg tadalafil dose that rescued functional muscle ischemia in our BMD patients is the same dose used clinically to treat men with erectile dysfunction and half the dose used to treat adult patients with pulmonary hypertension, the other Food and Drug Administration (FDA)-approved indication for tadalafil.

Patients do not develop tolerance when chronic PDE5A inhibition is used to treat erectile dysfunction or pulmonary hypertension; in contrast, with chronic nitroglycerin (a short-acting NO donor), healthy subjects quickly develop nitrate tolerance caused by reactive oxygen species that impair NO-mediated sympatholysis (44). The present data are consistent with our hypothesis that sarcolemmal nNOSμ plays an essential role in the normal modulation of sympathetic vasoconstriction in exercising human skeletal muscle and constitutes a first-in-human study of PDE5A inhibition as a putative new therapeutic strategy in BMD. Alleviating ischemic insult with PDE5A inhibitors to vulnerable dystrophin-deficient muscle membranes may reduce use-dependent muscle injury, thereby slowing disease progression. Also, PDE5A inhibition may be an important adjunct to exon skipping, which is under investigation for DMD to produce a truncated BMD-like dystrophin that may or may not restore sarcolemmal targeting of NOSμ (49,50). Repurposing PDE5A inhibitors could quickly transform clinical practice in muscular dystrophy, as no new drug development is needed. As loss of sarcolemmal nNOSμ is common in severe cases of acquired and inherited human neuromuscular diseases (29,47)—as well as in mouse models of steroid myopathy and (47) disuse atrophy (51)—the findings of our study may extend beyond BMD to a broader patient population.

That sympatholysis is impaired in contemporary patients with DMD confirms and extends our earlier observations made well over a decade ago before patients with DMD were treated with any medication. While glucocorticoids represent the first significant advance in medical management of DMD, our new data in glucocorticoid-treated patients show that chronic treatment with neither prednisone nor deflazacort protects dystrophic muscle from functional ischemia. While ACE-Is (lisinopril) and ARBs (losartan) are being used increasingly in boys with DMD as preemptive therapy to delay onset of heart failure, our data show that chronic treatment with neither lisinopril nor losartan restores sympatholysis in DMD, even though these drugs can reduce reactive oxygen species that destroy NO and thereby rescue sympatholysis in common acquired adult cardiovascular diseases such as hypertension and heart failure.

In contrast, the seminal finding of this study is that tadalafil rescues sympatholysis in boys with DMD, providing added putative benefit beyond that afforded by the current standard of care. The effect is marked, immediate, and dose-dependent. The lower dose used in our study is roughly equivalent to an adult dose of 20 mg, which is shown herein to alleviate functional ischemia in adults with BMD and is the highest dose approved by the FDA to treat erectile dysfunction. Our higher dose is roughly equivalent to the 40 mg dose approved by the FDA to treat adult pulmonary hypertension and the same dose most commonly used to treat pediatric pulmonary hypertension.

That sildenafil—which was the main PDE5 inhibitor used in the preclinical studies—and tadalafil, were virtually identical in their ability to restore sympatholysis in boys with DMD—despite having different chemical structures—strongly implicates PDE5 inhibition as the mechanism of action. Because PDE5 is a cGMP-specific phosphodiesterase, our data support the hypothesis that PDE5 inhibition boosts a residual NO-cGMP signal arising from cytosolic nNOS which, in the absence of dystrophin, is misplaced from the sarcolemma. That neither PDE5 inhibitor affected reflex vasoconstriction or blood flow in resting DMD skeletal muscle nor systemic blood pressure suggests that the exercise-specific action involves neither endothelial NOS-derived NO nor central inhibition of sympathetic outflow.

Moreover, that tadalafil normalized not only sympatholysis but also exercise-induced hyperemia—which was markedly blunted in the boys with DMD—provides further evidence for remarkably close clinical translation of seminal mdx mouse studies. This novel finding also suggests that NO released from exercising human skeletal muscle normally exerts a vasodilatory effect even in the absence of alpha-adrenergic stimulation. The blood flow data further suggest that a higher degree of PDE 5 inhibition may be required to fully restore exercise-induced hyperemia than sympatholysis. Whereas sympatholysis reflects attenuation of a neurogenic vasoconstrictor response specifically in the most distal skeletal muscle microvessels, exercise-induced hyperemia involves multiple vascular segments and multiple active vasodilator mechanisms that remain incompletely understood.

The present data provide compelling evidence that PDE5 inhibition is a treatment for patients with DMD, and advance the vascular hypothesis in DMD as a putative treatment target to slow disease progression and improve overall quality of life.

Various embodiments of the present invention are based, at least in part, on these finding.

Various embodiments of the present invention provide for a method of treating muscular dystrophy. In various embodiments, treating muscular dystrophy slows down disease progression of muscular dystrophy. In various embodiments, the method comprises administering a quantity of a phosodiesterase 5A (PDE5A) or a salt thereof to a subject in need of treatment for muscular dystrophy. In other embodiments, the method comprises providing a phosodiesterase 5A (PDE5A) inhibitor or a salt thereof; and administering a quantity of a PDE5A inhibitor to a subject in need of treatment for muscular dystrophy.

In certain embodiments, the muscular dystrophy is Becker muscular dystrophy (BMD). In other embodiments, the muscular dystrophy is Duchenne muscular dystrophy.

Various embodiments of the present invention provide for a method of restoring functional sympatholysis, comprising administering a quantity of a PDE5A inhibitor to a subject in need of restoring functional sympatholysis. In various embodiments, the method comprises providing a phosodiesterase 5A (PDE5A) inhibitor or a salt thereof; and administering a quantity of a PDE5A inhibitor to a subject in need of restoring functional sympatholysis. In various embodiments, the method restores functional sympatholysis at least or up to 1, 2, 3, 4, 5, 10, 15, 20, 25, 30, 35, 40, 45, 50, 60, 70, 80, 90, 95, 96, 97, 98, 99, or 100%.

Various embodiments of the present invention provide for a method alleviating ischemic insult to dystrophin-deficient muscle membranes. In various embodiments, the method comprises administering a quantity of a phosodiesterase 5A (PDE5A) or a salt thereof to a subject in need of alleviating ischemic insult to dystrophin-deficient muscle membranes. In other embodiments, the method comprises providing a phosodiesterase 5A (PDE5A) inhibitor or a salt thereof; and administering a quantity of a PDE5A inhibitor to a subject in need of alleviating ischemic insult to dystrophin-deficient muscle membranes.

Various embodiments of the present invention provide for a method to reduce use-dependent muscle injury. In various embodiments, the method comprises administering a quantity of a phosodiesterase 5A (PDE5A) or a salt thereof to a subject in need of reducing use-dependent muscle injury. In other embodiments, the method comprises providing a phosodiesterase 5A (PDE5A) inhibitor or a salt thereof; and administering a quantity of a PDE5A inhibitor to a subject in need of reducing use-dependent muscle injury.

Various embodiments of the present invention provide for a method to alleviate post-exercise hyperemia. In various embodiments, the method comprises administering a quantity of a phosodiesterase 5A (PDE5A) or a salt thereof to a subject in need of alleviating post-exercise hyperemia. In other embodiments, the method comprises providing a phosodiesterase 5A (PDE5A) inhibitor or a salt thereof; and administering a quantity of a PDE5A inhibitor to a subject in need of alleviating post-exercise hyperemia.

In various embodiments, the PDE5A inhibitor is selected from the group consisting of avanafil, lodenafil, mirodenafil, sildenafil, tadalafil, vardenafil, udenafil, zaprinast, UK357903 ((Pfizer) 1-ethyl-4-{3-[3-ethyl-6,7-dihydro-7-oxo-2-(2-pyridylmethyl)-2H-pyrazolo[4,3-d]pyrimidin-5-yl]-2-(2-methoxyethoxy)-5-pyridylsulphonyl}piperazine)) and combinations thereof.

In some embodiments, the PDE5A inhibitor is tadalafil or a salt thereof. In some embodiments, the PDE5A inhibitor is sildenafil or a salt thereof. In some embodiments, the PDE5A inhibitor is vardenafil or a salt thereof. In some embodiments, the PDE5A inhibitor is UK357903.

In some embodiments, the subject is human. In particular embodiments, the subject is an adult human. In particular embodiments, the subject is an adult, male human. For example, in embodiments wherein BMD is treated, it is treated in an adult, male human. In other particular embodiments, the subject is an adult, female human. In some embodiments, the subject is a human child (e.g., 17 years or younger). In particular embodiments, the subject is a human, male child. For example in embodiments wherein DMD is treated, it is treated in a human male child.

In some embodiments, the quantity of the PDE5A inhibitor is about 1.5, 2, 2.5, 3, 3.5, 4, 4.5, 5, 6, 7, 8, 9, 10, 11, 12, 13, 14, 15, 16, 17, 18, 10, 20, 21, 22, 23, 24, 25, 26, 27, 28, 29, 30, 35, 40, 45, 50, 55, 60, 65, 70, 75, 80, 85, 90, 95, 100, 110, 120, 125, 130, 140, or 150 mg.

In certain embodiments, the quantity of the PDE5A inhibitor is for a single dose therapy for use as needed; for example, prior to a period of heavy exertion, prior to a day when higher level of physical activity is expected. For example, a single dose can be administered to child before a day where he or she will be at an amusement park where it is expected that the child will be more active than normal. In these embodiments, the quantity of the PDE5A inhibitor can be about 0.5 to 1.0 mg/kg body weight or about 1.0 to 2.0 mg/kg body weight

In certain embodiments, the quantity of the PDE5A inhibitor is for continuous daily use. Quantities for continuous doses will be lower amounts; for example, 100 mg or lower, 50 mg or lower, or 20 mg or lower.

In some embodiments, wherein the PDE5A inhibitor is tadalafil or a salt thereof, the quantity is about 2.5, 5, 10, or 20 mg. In some embodiments, wherein the PDE5A inhibitor is tadalafil or a salt thereof, the quantity is about 1, 2, 2.5, 3, 4, 5, 6, 7, 8, 9, 10, 11, 12, 13, 14, 15, 16, 17, 18, 19, 20, 21, 22, 23, 24, 25, 30, 35, or 40 mg. In certain embodiments, the quantity is about 0.5 to 1.0 mg/kg body weight. In embodiments wherein a child is treated, the quantity of 0.5 to 1.0 mg/kg body weight will typically not exceed 40 mg even if that dose does not achieve 0.5 to 1.0 mg/kg body weight (e.g., in a heavy child). In certain embodiments, the quantity is about 20-40 mg per day; for example, for a single dose therapy for use as needed (e.g., prior to a period of heavy exertion, or prior to a day when higher level of physical activity is expected). In certain embodiments, the quantity is 20 mg or less per day for continuous daily use; for example, about 1.5, 2, 2.5, 3, 3.5, 4, 4.5, 5, 6, 7, 8, 9, 10, 11, 12, 13, 14, 15, 16, 17, 18, 10, 11, 12, 13, 14, 15, 16, 17, 18, 19 or 20 mg. In certain embodiments for continuous daily use, the quantity is about 0.2 to 0.7 mg/kg body weight, or about 0.3 to 0.6 mg/kg body weight. In particular embodiments, the quantity is about 0.2, 0.3, 0.4, 0.5, 0.6, or 0.7 mg/kg. In specific embodiments, the quantity is about 0.3 mg/kg. In specific embodiments, the quantity is about 0.6 mg/kg. In embodiments wherein a child is treated, the quantity of 0.2 to 0.7 mg/kg body weight, or about 0.3 to 0.6 mg/kg body weight will typically not exceed 40 mg even if that dose does not achieve 0.5 to 1.0 mg/kg body weight (e.g., in a heavy child). Typically, the dosage for the continuous daily use would not be more than 40 mg/day.

In some embodiments, wherein the PDE5A inhibitor is silendafil or a salt thereof, the quantity is about 20, 25, 50, or 100 mg. In some embodiments, wherein the PDE5A inhibitor is silendafil or a salt thereof, the quantity is about 10, 15, 20, 25, 30, 35, 40, 45, 50, 60, 70, 75, 80, 90, 100, 110, 120, 125, 130, 140, or 150 mg. In certain embodiments, the quantity is about 0.5 to 1.0 mg/kg body weight. In certain embodiments, the quantity is about 20-40 mg per day; for example, for a single dose therapy for use as needed (e.g., prior to a period of heavy exertion, or prior to a day when higher level of physical activity is expected). In certain embodiments, the quantity is 20 mg or less per day for continuous daily use; for example, about 1.5, 2, 2.5, 3, 3.5, 4, 4.5, 5, 6, 7, 8, 9, 10, 11, 12, 13, 14, 15, 16, 17, 18, 10, 11, 12, 13, 14, 15, 16, 17, 18, 19 or 20 mg. In certain embodiments for continuous daily use, the quantity is about 0.2 to 0.7 mg/kg body weight, or about 0.3 to 0.6 mg/kg body weight. In embodiments wherein a child is treated, the quantity of 0.2 to 0.7 mg/kg body weight, or about 0.3 to 0.6 mg/kg body weight will typically not exceed 40 mg even if that dose does not achieve 0.5 to 1.0 mg/kg body weight (e.g., in a heavy child). Typically, the dosage for the continuous daily use would not be more than 40 mg/day.

In some embodiments, wherein the PDE5A inhibitor is vardenafil or a salt thereof, the quantity is about 2.5, 5, 10, or 20 mg. In some embodiments, wherein the PDE5A inhibitor is vardenafil or a salt thereof, the quantity is about 1, 2, 2.5, 3, 4, 5, 6, 7, 8, 9, 10, 11, 12, 13, 14, 15, 16, 17, 18, 19, 20, 21, 22, 23, 24, 25 or 30 mg. In certain embodiments, the quantity is about 0.5 to 1.0 mg/kg body weight. In certain embodiments, the quantity is about 20-40 mg per day; for example, for a single dose therapy for use as needed (e.g., prior to a period of heavy exertion, or prior to a day when higher level of physical activity is expected). In certain embodiments, the quantity is 20 mg or less per day for continuous daily use; for example, about 1.5, 2, 2.5, 3, 3.5, 4, 4.5, 5, 6, 7, 8, 9, 10, 11, 12, 13, 14, 15, 16, 17, 18, 10, 11, 12, 13, 14, 15, 16, 17, 18, 19 or 20 mg. In certain embodiments for continuous daily use, the quantity is about 0.2 to 0.7 mg/kg body weight, or about 0.3 to 0.6 mg/kg body weight. In embodiments wherein a child is treated, the quantity of 0.2 to 0.7 mg/kg body weight, or about 0.3 to 0.6 mg/kg body weight will typically not exceed 40 mg even if that dose does not achieve 0.5 to 1.0 mg/kg body weight (e.g., in a heavy child). Typically, the dosage for the continuous daily use would not be more than 40 mg/day.

In certain embodiments, the quantity of the PDE5A inhibitor is administered 1, 2, 3, 4, or 5 times a day. In certain embodiments, the quantity of the PDE5A inhibitor is administered once per day (e.g., tadalafil is administered once per day). In certain embodiments, the quantity of the PDE5A inhibitor is administered 3 times per day or over a period of 3 times per day (e.g., sildenafil is administered 3 times per day)

In some embodiments, the quantity of the PDE5A inhibitor is administered prior to a planned period of physical exertion. In some embodiments, the quantity of the PDE5A inhibitor is administered about 4, 3, 2, or 1 hours prior to a planned period of physical exertion. In certain embodiments, the quantity of the PDE5A is administered about 2.5, 3, or 3.5 hours prior to a planned period of physical exertion. In certain embodiments, the quantity of the PDE5A is administered about 3 hours prior to a planned period of physical exertion.

In some embodiments, continuous daily administration can be done with the quantity of the PDE5A inhibitor taken every morning upon awakening. In some embodiments, the quantity of the PDE5A inhibitor taken every day upon awakening for the day; for example, in subjects who are employed in later shifts, their awakening for the day may be in the afternoon. Thus, in those subjects, the daily quantity is taken in the afternoon upon awakening.

In some embodiments, the quantity of the PDE5A inhibitor is administered prior to physical activity.

In certain embodiments, the quantity of the PDE5A inhibitor is administered for 1, 2, 3, 4, 5, 6, or 7 days. In certain embodiments, the quantity of the PDE5A inhibitor is administered for 1, 2, 3, 4, 5, 6, 7, or 8 weeks. In certain embodiments, the quantity of the PDE5A inhibitor is administered for 1, 2, 3, 4, 5, 6, 7, 8, 9, 10, 11, or 12 months. In certain embodiments, the quantity can be administered indefinitely.

Various embodiments of the present invention provide for method of treating Becker muscular dystrophy (BMD), comprising: providing about 20 mg of tadalafil or a salt thereof; and administering the about 20 mg of tadalafil or the salt thereof to an adult human subject in need of treatment for BMD. In various embodiments, the adult human subject is male.

Various embodiments of the present invention provide for method of treating Duchenne muscular dystrophy (DMD), comprising: providing about 0.5-1.0 mg/kg of tadalafil or a salt thereof; and administering the about 0.5-1.0 mg/kg of tadalafil or the salt thereof to an human child subject in need of treatment for DMD. In various embodiments, the human child subject is male.

Various embodiments of the present invention provide for method of treating Duchenne muscular dystrophy (DMD) in a human child, comprising: providing about 0.3-0.6 mg/kg of tadalafil or a salt thereof; and administering the about 0.3-0.6 mg/kg of tadalafil or the salt thereof to the human child in need of treatment for DMD. In various embodiments, the human child subject is male. In particular embodiments, about 0.3 mg/kg of tadalafil or a salt thereof is provided and administered. In particular embodiments, about 0.6 mg/kg of tadalafil or a salt thereof.

In various embodiments, the present invention provides pharmaceutical compositions including a pharmaceutically acceptable excipient along with a therapeutically effective amount of the PDE5A inhibitor. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. For additional guidance, see Remington: The Science and Practice of Pharmacy (Gennaro ed. 20th edition, Williams & Wilkins PA, USA) (2000).

Kits

The present invention is also directed to a kit to for the treatment of muscular dystrophy. The kit is useful for practicing the inventive method of treating muscular dystrophy. The kit is an assemblage of materials or components, including at least one of the inventive compositions. Thus, in some embodiments the kit contains a composition including a PDE5A inhibitor as described above.

The exact nature of the components configured in the inventive kit depends on its intended purpose. For example, some embodiments are configured for the purpose of treating muscular dystrophy. In one embodiment, the kit is configured particularly for the purpose of treating human subjects. In another embodiment, the kit is configured particularly for the purpose of treating adult, human subjects. In another embodiment, the kit is configured particularly for the purpose of treating children. In another embodiment, the kit is configured particularly for the purpose of treating DMD. In another embodiment, the kit is configured particularly for the purpose of treating BMD. In another embodiment, the kit is configured particularly for the purpose of providing continuous daily use dosages. In another embodiment, the kit is configured particularly for the purpose of providing as needed use dosages. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat muscular dystrophy, to treat BMD, to treat DMD, to alleviate ischemic insult to vulnerable dystrophin-deficient muscle membranes, to reduce use-dependent muscle injury, to slow down disease progression of muscular dystrophy. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as the PDE5A inhibitors described herein and the like. The packaging material is constructed by well-known methods, preferably to provide a sterile, contaminant-free environment. The packaging materials employed in the kit are those customarily utilized in therapeutic treatment. As used herein, the term “package” refers to a suitable solid matrix or material such as glass, plastic, paper, foil, and the like, capable of holding the individual kit components. Thus, for example, a package can be a plastic bottle used to contain suitable quantities of an composition containing a PDE5A inhibitor. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Becker Muscular Dystrophy

Subjects

We studied 10 ambulatory male patients with BMD, 18 to 55 years of age, with normal LVEF, and seven healthy male controls matched for age and BMI. All patients had a pre-existing clinical diagnosis of BMD, which we confirmed with direct sequencing analysis of the dystrophin gene (University of Utah Genome Center, Salt Lake City, Utah).

Potential subjects (both cases and controls) were excluded from study if they had: a history of hypertension or measured blood pressure >140/90 mmHg; diabetes mellitus; heart failure by history, physical examination, elevated brain natriuretic peptide (BNP), or LVEF<50% by echocardiography; nocturnal ventilator support; serum creatinine ≧1.5 mg/d; any history of substance abuse (including alcohol) or other psychiatric illness; or contraindications to tadalafil (use of nitrates, α-adrenergic blockers, other PDE5A inhibitors, or potent inhibitors of cytochrome P450 3A4).

The study was approved by the Institutional Review Board at Cedars-Sinai Medical Center and each subject gave informed written consent to participate. A waiver for an investigational new drug application was granted by the FDA to use tadalafil in this trial. The study was registered with clinicaltrials.gov (identifier NCT01070511).

Direct Sequencing Analysis of the Dystrophin Gene

Mutation screening of the dystrophin gene was performed by multiplex ligation-dependent probe amplification (MLPA) of genomic DNA isolated from whole blood (52). The test surveyed single or multi-exon duplication in the dystrophin gene, using MLPA to screen for duplications in all 79 coding exons of the major mRNA transcript isoform in muscle (Dp427m, reference mRNA transcript NM 004006). MLPA was performed as described (53) with the P034 and P035 kits from MRC-Holland. These MLPA reactions were performed on genomic DNA. The extent of exonic deletion was confirmed by polymerase chain reaction (PCR) (54) using four independent primer pairs to verify the absence of each limiting exon.

Hemodynamic Measurements

Subjects were studied in the supine position. Heart rate was measured continuously by electrocardiography and BP by automated oscillometric sphygmomanometry (Welch Allyn Vital Signs Monitor 300 Series, Skaneateles Falls, N.Y.).

Skeletal Muscle Oxygenation in NIR Spectroscopy

Forearm muscle oxygenation was measured with near-infrared (NIR) spectroscopy, which is based on the principle that laser light with wavelengths in the 700-900 nm range easily penetrates skeletal muscle, and is absorbed by the iron-porphyrin moieties in hemoglobin and myoglobin. Changes in NIR light absorption are proportional to changes in the relative concentrations of oxygenated hemoglobin and myoglobin (HbO₂ and MbO₂). Because of their nearly identical absorption spectra, individual contributions of HbO₂ and MbO₂ cannot be determined. The NIR signals reflect changes in oxygenation occurring mainly in the microvasculature, because vessels greater than one millimeter in diameter are maximal absorbers of photons due to the high extinction coefficient of blood. Thus, NIR spectroscopy provides continuous measurement of the adequacy of tissue oxygen delivery relative to its use.

To monitor the tissue absorption of NIR light, five optical fiber bundles (four emitting bundles and one detector bundle connected to a photomultiplier tube) housed in a customized flexible rubber casing were placed with adhesive on the skin over the flexor digitorum profundus muscle, the main muscle recruited during handgrip. Four fixed emitter-detector distances (1.5, 2.0, 2.5, and 3.0 cm) allowed direct calculation and, thus subtraction, of scatter from skin and subcutaneous non-muscle tissue (55). Each emitting bundle contained two laser-diode light sources, one at 830 nm, the wavelength at which the oxygenated and deoxygenated Hb/Mb species exhibit similar absorption coefficients, and the other at 690 nm, the wavelength at which light is absorbed primarily by the deoxygenated species. The difference between absorption at the two wavelengths is the HbO₂+MbO₂ (56). The NIR signals were sampled at a rate of five Hz, converted to HbO₂+MbO₂ concentration using validated algorithms, displayed as the running average of 50 consecutive samples, and stored digitally for analysis (OxiplexTS, ISS, Inc. Champaign, Ill.). Before each experiment, absorption and scattering coefficients at each wavelength were calibrated against an external standard. After each experiment, a cuff was inflated on the upper arm to suprasystolic pressure of 250 mmHg to establish the “total labile signal” (TLS, the difference between the baseline and nadir in muscle tissue oxygenation). Changes in forearm tissue oxygenation were expressed as a percentage of TLS.

Handgrip Exercise

Handgrip exercise was performed using a dynamometer (Smedley Hand Dynamometer modified by Stoelting, Wood Dale, Ill.). To determine MVC, each subject was asked to grip the dynamometer as hard as possible. Force output was displayed on a computer screen to provide visual feedback for subjects. Subjects performed intermittent isometric handgrip (20 handgrips/minute, 50% duty cycle) at 20% MVC for seven minutes. This mild level of handgrip exercise alone does not activate sympathetic outflow to skeletal muscle (13,57).

Reflex Sympathetic Activation with Lower Body Negative Pressure

The subject's lower body was enclosed in a negative pressure chamber to the level of the iliac crest as previously described (13). The pressure in the chamber was measured by a Statham transducer (Gould Inc., Oxnard, Calif.). LBNP at −20 mmHg simulates mild orthostatic stress (i.e., transition from the supine to the seated position). This technique mainly unloads the cardiopulmonary baroreceptors, producing highly reproducible reflex increases in sympathetic vasoconstrictor drive to the skeletal muscle vasculature without changing systemic arterial pressure (13,58-60).

Functional Sympatholysis

To measure exercise-induced attenuation of reflex vasoconstriction (i.e., functional sympatholysis), LBNP was applied (a) at rest, and then (b) superimposed on mild handgrip exercise. Reflex vasoconstriction was measured as the LBNP-induced decrease in forearm muscle oxygenation by NIR spectroscopy; this approach provides a valid measure of reflex neurogenic vasoconstriction under these conditions (13).

The BP, heart rate, handgrip force, and NIR signals were recorded in response to two minutes of LBNP applied at rest and during the third to fifth minutes of the seven-minute exercise period.

Protocol for the Case-Control Study

All subjects were screened for eligibility with a clinical examination, comprehensive blood chemistry panel, ECG, and echocardiogram. Patients with BMD also had blood drawn for CK, BNP, and DNA. In all eligible subjects, functional sympatholysis was measured in both arms (random order) on two separate days; all four measurements were averaged to calculate each subject's mean value.

Protocol for the Tadalafil Acute Treatment Trial

All BMD patients who completed the case-control protocol also completed the tadalafil treatment trial, which utilized a randomized, placebo-controlled, double-blind, crossover design with a two-week washout period before cross-over (to account for the 17.5-hour elimination half-life of tadalafil). Patients received a 10 mg test dose of oral tadalafil or placebo the night before the study (to test for adverse events) and, on the next morning, a 20 mg tablet of tadalafil or placebo at least three hours (to allow for peak tadalafil absorption) before measurement of functional sympatholysis (in both arms). The order of tadalafil/placebo was random. The research pharmacist placed tadalafil tablets or lactose powder (placebo) in opaque capsules to blind patients and investigators, both for the test dose and study dose of drug or placebo. Patients were queried about potential tadalafil-specific side-effects both for safety monitoring and to assess whether blinding was maintained. tadalafil blood levels were measured using high performance liquid chromatography/tandem mass spectrometry (NMS Labs Willow Grove, Pa.).

Immunohistochemistry

Needle muscle biopsies from two patients (P5 and P9) were obtained using standard technique and mounted in Optimal Cutting Temperature (OCT) compound and frozen in isopentane cooled in liquid nitrogen. Cryosections (6 μm) were cut and mounted onto SuperFrost® Plus slides. Sections were incubated with monoclonal antibodies for nNOS (NCL-NOS-1, Novocastra, 1:400), and dystrophin C-terminus (NCL-DYS2, Novocastra, 1:1000). Immunodetection was carried out using a sensitive detection protocol (X-Cell-Plus HRP Detection—Menapath MPXCPDAB-U100, according to the manufacturer's instructions). Sections were visualized with Liquid stable DAB (Menapath), counterstained in Carazzi's hematoxylin, dehydrated and permanently mounted. Primary antibody was omitted in negative controls. Patient biopsies were compared to a stored muscle sample with normal histology and protein expression previously obtained from a healthy individual. Hematoxylin & Eosin staining was performed with the standard protocol.

Statistics

Baseline characteristics of patients and controls were compared using Student's t test with the Welch variation for degrees of freedom applied to unequal variances. Exercise-induced attenuation of reflex vasoconstriction (functional sympatholysis) was assessed by comparing the LBNP-induced AHbO₂+MbO₂ (% TLS) at rest vs. the LBNP-induced decrease in AHbO₂+MbO₂ during handgrip. For each subject in the case-control study, the mean data from both arms on each study day were meaned to derive a single value for the LBNP response at rest and for the LBNP response during handgrip. These data were assessed for normality with the Kolmogorov-Smirnov test and for homogeneity of variances with Levene's test. We tested for group differences in AHbO2+MbO2 during LBNP using a linear mixed effects model, with fixed effects for group (BMD patients or controls) and exercise condition (rest or handgrip) and random effects for individual subjects. In the tadalafil treatment trial, data from both arms were meaned to obtain patient-specific values for LBNP responses during placebo and during tadalafil. The main analysis was a linear mixed-effects model appropriate for a two-period and two-treatment randomized crossover design, containing fixed effects for sequence, period, drug treatment, exercise condition, baseline values, and subject within sequence as a random effect. Testing for carryover effects (using the sequence effect) and period effects was carried out; neither effect was statistically significant. The threshold of significance for the carryover effect was 0.1. For all other variables, two-sided P values of less than 0.05 were considered to indicate statistical significance. All analyses were performed in R, version 2.13.1. Data are expressed as mean±standard error (SEM).

Immunoblot

Whole-muscle lysate was extracted and Western Blot was performed according to a previously published protocol (61). The membranes were probed with antibodies for the dystrophin C-terminus (1:100), dystrophin rod domain (NCL-DYS1, Novocastra, 1:300), and anti-desmin (Dako, 1:400) as indicators of muscle proteins. Membranes were then incubated in horseradish peroxidase (HRP) conjugated rabbit anti-mouse immunoglobulin-G (IgG) secondary antibody (Dako, 1:300), followed by chemiluminescent detection (SuperSignal West Pico Chemiluminescent Substrate, Pierce).

Results

Of 15 patients screened for eligibility, five men were excluded: two were too weak to perform handgrip, one had reduced left ventricular ejection fraction (LVEF), one had hypertension, and one did not have BMD by mutational analysis. No patients with BMD were on nocturnal ventilator support.

Characteristics of the Study Groups

Baseline characteristics and indices of disease severity of the individual patients with BMD are shown in Table 1. All patients were ambulatory, though the most severely affected patient P10 often uses a wheelchair. Six of the ten patients (P1-P6) had exonal deletions encoding the spectrin-like repeats of dystrophin implicated in sarcolemmal targeting of nNOSμ (11,33,34). All ten subjects had elevated creatine kinase (CK) levels as expected.

TABLE 1
Baseline characteristics of patients with Becker muscular dystrophy

| | | | | | Maximum | | | | |
|---|---|---|---|---|---|---|---|---|---|
| | | | | | voluntary | | | | |
| | | | Age at | Age at loss | contraction | | | | |
| | | Age, | onset of | of ability | (grip strength) | | CK | Frequency of | |
| | Exon | at study, | symptoms, | to run, | Rt arm, | Lt arm, | level, | wheelchair/ | |
| | Deletion | years | years | years | kg | kg | IU/L | scooter use | Medications |
| P1 | 45-48 | 32 | 5 | 18 | 32 | 22 | 1883 | Never | none |
| P2 | 45-48 | 33 | 5 | 22 | 23 | 29 | 1247 | Never | none |
| P3 | 45-48 | 42 | 30 | 30 | 42 | 39 | 719 | Never | none |
| P4 | 45-55 | 38 | 12 | 26 | 29 | 23 | 2006 | Never | none |
| P5 | 45-48 | 42 | 28 | 32 | 29 | 33 | 1453 | Rarely | none |
| P6 | 48-49 | 38 | 25 | 27 | 35 | 33 | 2058 | Never | none |
| P7 | 67 | 50 | 32 | 38 | 36 | 42 | 2247 | Rarely | oxybutynin |
| P8 | 20-29 | 30 | 12 | 15 | 13 | 10 | 3745 | Never | deflazacort |
| P9 | 14-44 | 50 | 33 | 35 | 21 | 17 | 707 | Never | none |
| P10 | 3-7 | 19 | 6 | 8 | 13 | 12 | 2448 | Often | none |

P, patient;
CK, creatine kinase

As shown in Table 2, patients and controls were matched for age, body mass index (BMI), blood pressure (BP), heart rate, and LVEF. BMD patients, as expected, had lower grip strength (as measured by handgrip maximum voluntary contraction, MVC) in both arms.

TABLE 2
Baseline comparison of the two study groups.

| | | | | MVC | MVC | | | HR, | |
|---|---|---|---|---|---|---|---|---|---|
| | | Age, | BMI, | right arm, | left arm, | SBP, | DBP, | beats | LVEF, |
| Group | n | years | kg/m | kg | kg | mmHg | mmHg | per min | % |
| Patients with BMD | 10 | 37 ± 3 | 25.0 ± 1.6 | 27 ± 3* | 26 ± 3* | 118 ± 3 | 78 ± 3 | 79 ± 5 | 60.7 ± 1.8 |
| Healthy controls | 7 | 34 ± 3 | 25.0 ± 1.7 | 54 ± 2 | 47 ± 3 | 114 ± 2 | 71 ± 3 | 78 ± 2 | 65.7 ± 1.3 |

BMI, body mass index;
MVC, maximum voluntary contraction;
SBP, systolic blood pressure;
DBP, diastolic blood pressure;
HR, heart rate;
LVEF, left ventricular ejection fraction.
*P < 0.05 versus controls.

Functional Sympatholysis is Impaired in BMD

To compare muscle blood regulation in patients vs. controls, we assessed functional sympatholysis, which refers to exercise-induced attenuation of reflex sympathetic vasoconstriction—a protective mechanism that normally ensures perfusion of metabolically active skeletal muscle. Functional sympatholysis was assessed by applying the same reflex sympathetic stimulus—lower body negative pressure (LBNP), which simulates mild orthostatic stress and engages cardiac baroreceptors to trigger reflex sympathetic activation to the skeletal muscle circulation—with the subjects' forearm muscles either resting or performing light rhythmic handgrip exercise. Sympathetic constriction of skeletal muscle microvessels was measured as the decrease in forearm muscle oxygenation—oxygenated hemoglobin plus oxygenated myoglobin (HbO₂+MbO₂) as measured by near infrared spectroscopy—in response to LBNP.

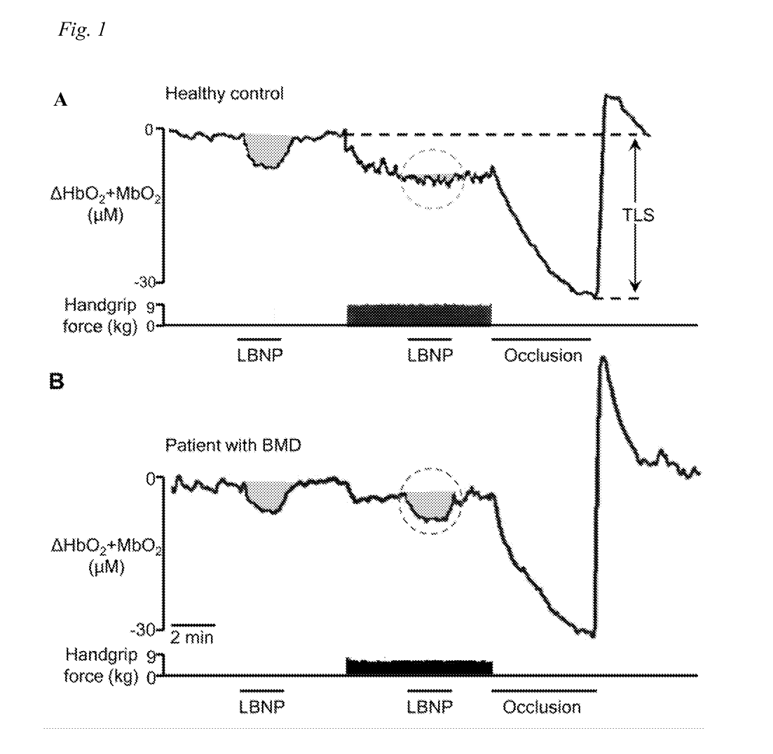

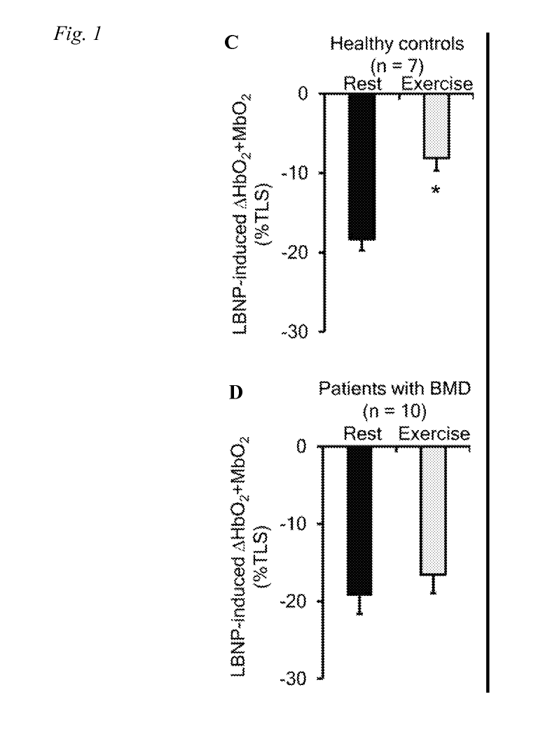

In resting forearm muscle, LBNP evoked comparable decreases in forearm muscle oxygenation (HbO₂+MbO₂) in BMD patients and healthy control individuals, indicating comparable reflex vasoconstriction (FIG. 1A,B). During the increased oxygen consumption with handgrip exercise alone, muscle oxygenation decreased in both groups, quickly reaching a new steady state level. When LBNP was superimposed on handgrip in healthy controls, the reflex decrease in muscle oxygenation was attenuated by 60±8% (AHbO2+MbO2: −18±1% at rest vs. −7±2% during handgrip, P<0.01), indicating functional sympatholysis. However, no such attenuation was seen in the patients with BMD (AHbO₂+MbO₂: −19±2% at rest vs. −17±2% during handgrip, P>0.10), indicating impaired sympatholysis and thus functional muscle ischemia (FIG. 1C,D).

Tadalafil Restores Functional Sympatholysis in BMD

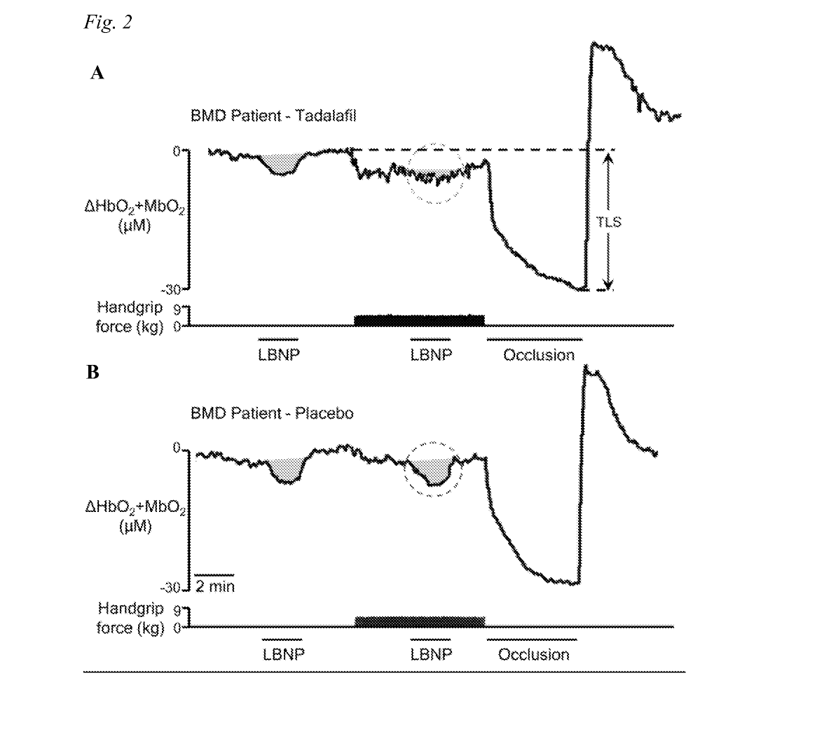

Then, to test if tadalafil can restore sympatholysis in BMD, all 10 patients completed a doubleblind randomized cross-over trial. Patients were randomly assigned by a research pharmacist to have sympatholysis assessed after receiving either a single 20 mg capsule of tadalafil or a placebo capsule; after a two-week washout period, sympatholysis was re-assessed after the subject received the other treatment (the tadalafil group was switched by the research pharmacist to receive placebo and vice-versa). As shown in FIG. 2, tadalafil treatment restored functional sympatholysis in BMD patients. When LBNP was superimposed on handgrip after tadalafil treatment, the reflex decrease in muscle oxygenation was attenuated by 52±12% (AHbO₂+MbO₂: −17±2% at rest vs. −9±2% during handgrip, P<0.01). In contrast, BMD patients treated with a placebo showed no effect: LBNP evoked comparable decreases in muscle oxygenation at rest and during exercise (AHbO2+MbO2: −18±2% at rest vs. −17±2% during handgrip; P>0.1).

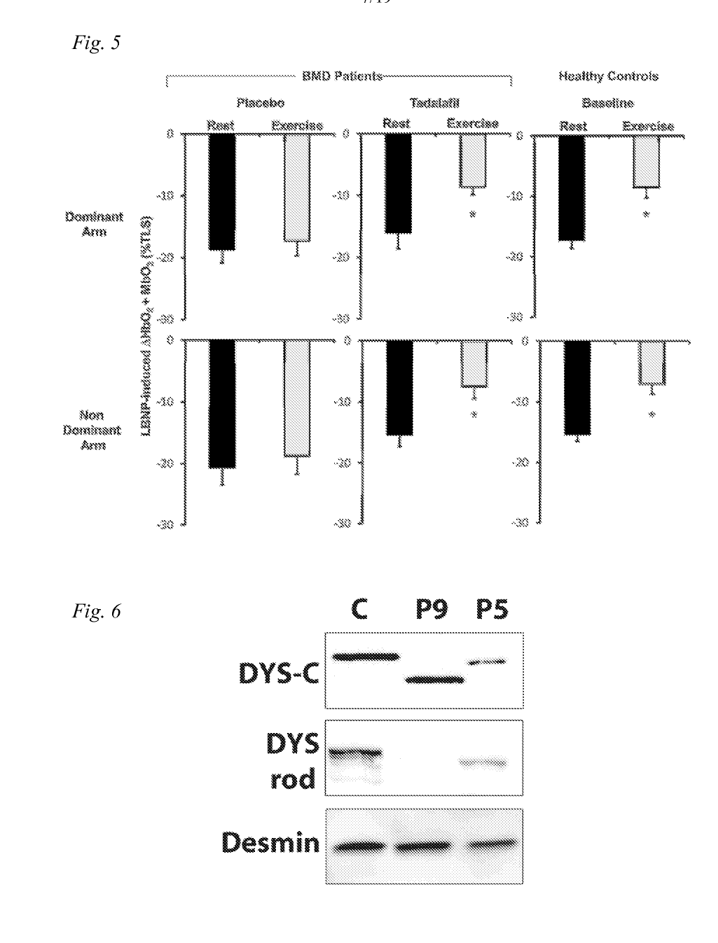

Comparing each patient's LBNP response during handgrip in the placebo arm of the treatment trial with that during the case-control study, the intraclass correlation was r=0.80, demonstrating reproducibility of impaired functional sympatholysis in the absence of active drug treatment. Furthermore, the degree of functional sympatholysis in tadalafil-treated patients was indistinguishable from normal (healthy controls, P>0.1). Hand dominance had no effect on the results of sympatholysis experiments (FIG. 5).

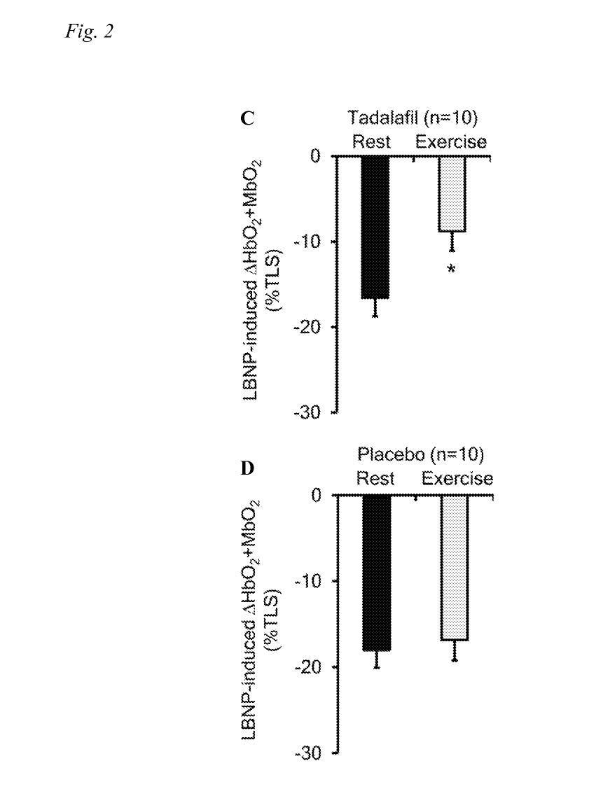

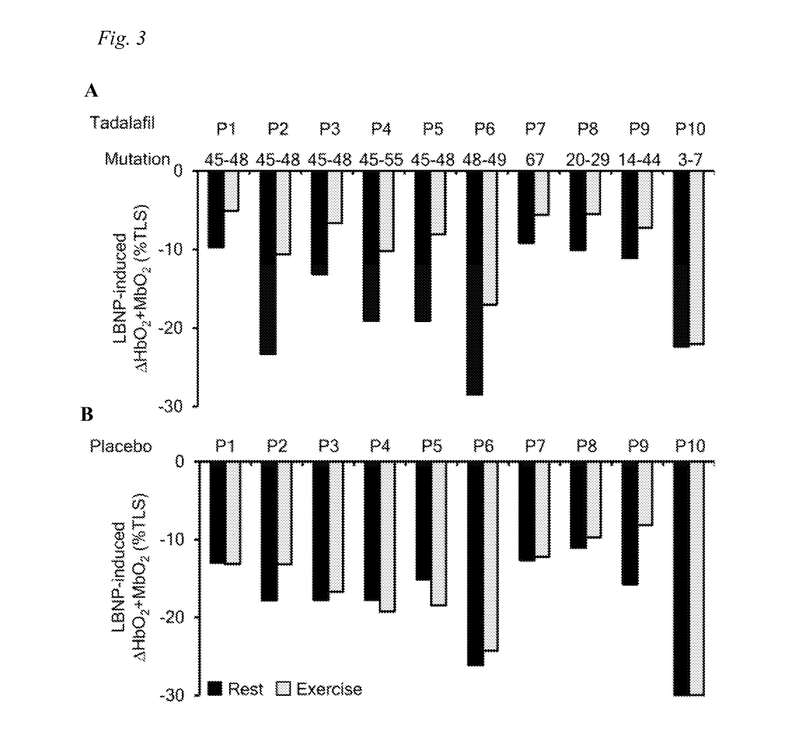

FIG. 3 shows the patient-specific responses to tadalafil and to placebo. Nine of ten patients with BMD had sympatholysis after tadalafil, whereas only one patient (P9) had sympatholysis with placebo. Deletion of exons 14-44 in this patient would be expected to result in a truncated dystrophin protein that could still target nNOSμ to the sarcolemma (35).

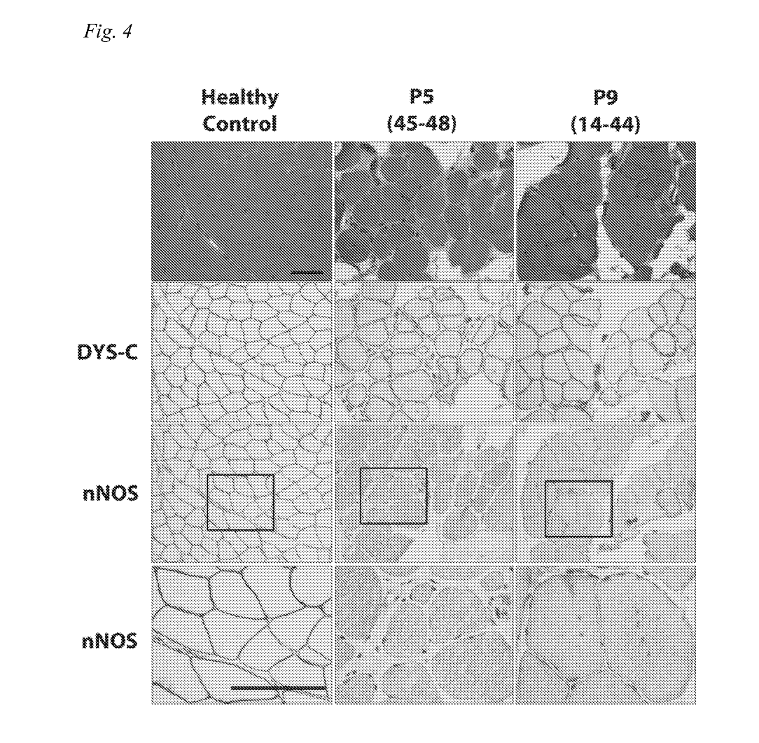

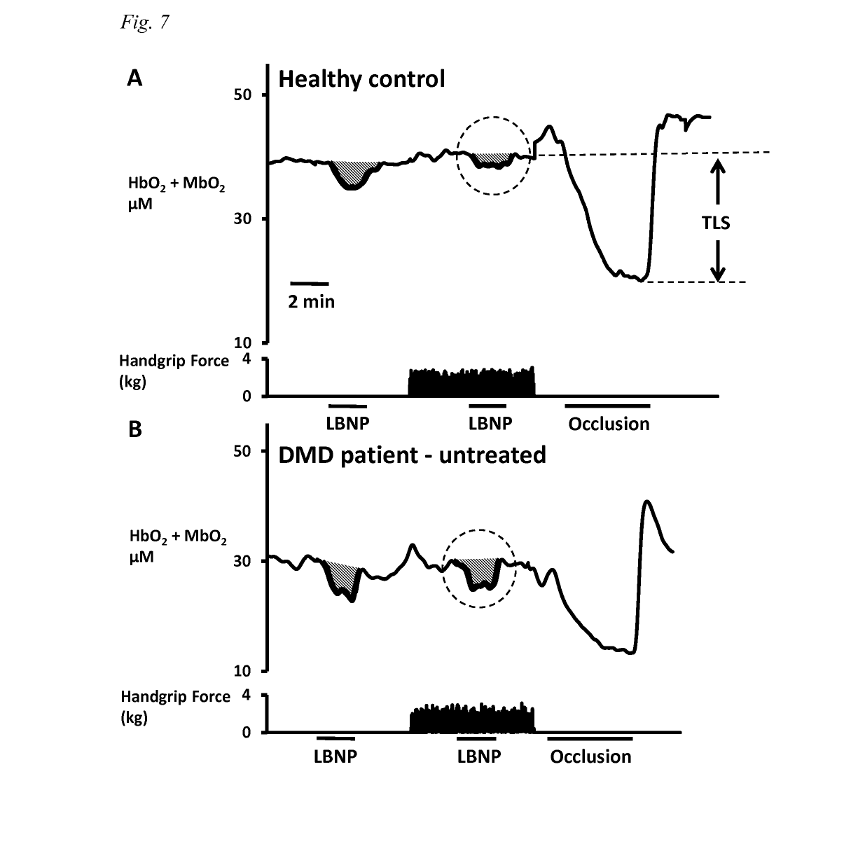

FIG. 4 shows the results of immunohistochemistry experiments performed on muscle biopsy samples from P9 and, by comparison, one of the patients (P5) with deletion of exons 45-48. Sarcolemmal nNOS expression indeed was detected in P9 (at a reduced level) but not in P5 (as expected); cytoplasmic nNOS expression was detected in both patients. Dystrophin Cterminus staining on sections was similar to normal muscle in both patients, but immunoblot analysis showed truncated or decreased dystrophin characteristic of BMD in both patients (FIG. 6). Peak tadalafil blood concentrations ranged from 170-310 ng/ml (mean: 260±13 ng/ml). In the one patient (P10) with no sympatholysis after tadalafil, the peak blood concentration of tadalafil was 300 ng/ml. tadalafil had no effect (P>0.05) on systolic blood pressure (116±3 vs. 114±4 mmHg, pre- vs. post-tadalafil), diastolic blood pressure (72±2 vs. 73±3 mm Hg), or heart rate (84±4 vs. 80±4 beats per minute). There were no adverse events or side-effects with tadalafil; specifically, no patients experienced spontaneous penile erection, flushing, or visual disturbance either with the test dose or study dose of either tadalafil or placebo.

### Example 2

### Duchenne Muscular Dystrophy

Subjects

We recruited ambulatory boys (ages 8-13 years) with DMD, who had normal left ventricular ejection fraction (>50%), and healthy age-matched boys to serve as healthy controls. All patients had a preexisting clinical diagnosis of DMD.

Potential subjects (both cases and controls) were excluded from the study if they had a history of hypertension or measured BP>120/80 mmHg; diabetes mellitus; heart failure by history, physical examination, or left ventricular ejection fraction <50% by two-dimensional echocardiography; were using nocturnal ventilator support; or had any contraindications to phosphodiesterase inhibitors (use of nitrates, α-adrenergic blockers, other PDE5A inhibitors, or potent inhibitors of cytochrome P450 3A4).

Hemodynamic Measurements

Subjects were studied in the supine position. Heart rate was measured continuously by electrocardiography, and blood pressure measured by automated oscillometric sphygomanometry using a child's cuff (Welch Allyn Vital Signs Monitor 300 Series).

Skeletal Muscle Oxygenation by Near-Infrared Spectroscopy

Forearm muscle oxygenation was measured with near-infrared (NIR) spectroscopy, as previously described.^{4B} Briefly, optodes were placed over the belly of the flexor digitorum profundus, the main muscle recruited during handgrip. The optodes were housed in a customized flexible rubber casing, ensuring a fixed and invariant position of the optodes relative to each other, and was secured to the skin with adhesive. The optodes were covered with an optically dense, black vinyl sheet, to minimize interference from external light or loss of NIR transmitted light. The forearm was wrapped with an elastic bandage to minimize movement of the optodes.

The NIR signal was sampled at a rate of 5 Hz, converted to HbO₂+MbO₂ concentration with validated algorithms, displayed as the running average of 50 consecutive samples, and stored digitally for analysis (OxiplexTS, ISS Inc.). Before each experiment, absorption and scattering coefficients were calibrated against an external standard. After each experiment, a cuff was inflated on the upper arm to supra-systolic pressure of 200 mmHg to establish the total labile signal (TLS, the difference between the baseline and nadir in muscle tissue oxygenation). Changes in forearm tissue oxygenation were expressed as a percentage of TLS.

Brachial Artery Blood Flow by Doppler Ultrasound

Brachial artery mean blood velocity (MBV) was measured from the exercising arm using pulsed-Doppler ultrasonography (Siemens iE33). Data were acquired continuously with a 9-MHz probe with a 60° angle of insonation, placed on the skin surface distal to the axilla. The ultrasound gate was optimized to ensure complete insonation of the entire vessel cross-section with constant intensity. The Doppler audio signal was converted to a real-time flow velocity signal using a validated Doppler audio converter,^{8B} and recorded using a PowerLab data acquisition system (ADInstruments, CO). Brachial artery diameter was measured by B-mode imaging in triplicate at rest. Previous reports have demonstrated that brachial artery diameter does not change from the resting value over a wide range of handgrip exercise. Brachial artery blood flow was calculated as MBV (cm/s)·πr²·60, where r is radius of the brachial artery.

Handgrip Exercise

Handgrip exercise was performed with a dynamometer (Smedley Hand Dynamometer modified by Stoelting). To determine maximal voluntary contraction (MVC), each subject was asked to grip the dynamometer as hard as possible. Force output was displayed on a computer screen to provide visual feedback for subjects. Subjects performed intermittent isometric handgrip (20 handgrips per minute, 50% duty cycle) at 20% MVC for 7 minutes. This mild level of handgrip exercise alone does not activate sympathetic outflow to skeletal muscle.^{9B, 10B}

Reflex Sympathetic Activation with Lower Body Negative Pressure

The subjects lower body was enclosed in a negative pressure chamber to the level of the iliac crest as previously described.^{9B} The pressure of the chamber was measured by a Statham transducer (Gould Inc.). Lower body negative pressure (LBNP) at −20 mmHg simulates mild orthostatic stress (that is, transition from the supine to the seated position). This technique mainly unloads the cardiopulmonary baroreceptors, producing highly reproducible reflex increases in sympathetic vasoconstrictor drive to the skeletal muscle vasculature without changing systemic blood pressure.^{9B}

Functional Sympatholysis

The subjects' lower body was enclosed in a negative pressure chamber to the level of the iliac crest as previously described. The pressure in the chamber was measured by a Statham transducer (Gould Inc.). Lower body negative pressure (LBNP) at −20 to −30 mmHg simulates mild orthostatic stress caused by transition from the supine to the seated position. This technique mainly unloads the low-pressure cardiopulmonary baroreceptors, producing highly reproducible reflex increases in sympathetic vasoconstrictor drive to the skeletal muscle vasculature without changing systemic blood pressure. To measure exercise-induced attenuation of reflex vasoconstriction (i.e., functional sympatholysis), the LBNP was (i) applied at rest, and then (ii) superimposed on mild rhythmic handgrip at 20% MVC. Reflex vasoconstriction was measured as the LBNP-induced decrease in forearm muscle oxygenation by NIR spectroscopy. Using the pressure inside the LBNP chamber as a trigger, the Powerlab software was programed to mean the HbO₂+MbO₂ signal for 20 seconds before the onset of LBNP and for 20 seconds before the offset of LBNP; the difference between these mean values was taken as the LBNP-induced change in forearm muscle tissue oxygenation (20). Blood pressure, heart rate, and handgrip force were also recorded in response to 2 minutes of LBNP applied at rest and during the third to fifth minutes of the 7-minute exercise protocol.

Exercise-Induced Hyperemia

Brachial artery blood flow was measured at rest and for 60 seconds post-exercise to evaluate exercise induced changes in skeletal muscle blood flow, defined as the percentage increase in brachial artery blood flow from rest to post-exercise. Vascular conductance was calculated as forearm blood flow divided by mean arterial pressure.

Pharmacokinetic Study and Drug Blood Levels

Serum blood samples for pharmacokinetic determinations were collected from patients at time 0-hr, 0.25-hr, 0.5-hr, 1-hr, 2-hr, 4-hr, 8-hr, and 24-hr following each treatment (sildenafil and tadalafil) and each dose (0.5 vs. 1.0 mg/kg). Blood levels were measured with high-performance liquid chromatography-tandem mass spectrometry (NMS Labs).

Protocol—Case-Control Study

All subjects were screened for eligibility with a clinical examination, 12-lead electrocardiogram, and two-dimensional transthoracic echocardiogram. Functional sympatholysis was measured using the left arm.

Protocol—Phosphodiesterase Inhibition

Patients received either sildenafil or tadalafil in an open-label crossover fashion with a 2-week washout period before crossover (to account for the 17.5-hour elimination half-life of tadalafil). Patients received 0.5 mg/kg of oral sildenafil or tadalafil on day 1, followed by 1.0 mg/kg of sildenafil or tadalafil on day 2. Patients then crossed over 2 weeks later for the opposite drug. Experiments were performed 1 hour (sildenafil) or 3 hours (tadalafil) after oral treatment to match the experiment with the peak blood level. Patients were queried about potential side-effects throughout the study visit.

Statistics

Baseline characteristics of patients and controls were compared using Student's t test. Exercise-induced attenuation of reflex vasoconstriction (functional sympatholysis) was assessed by comparing the LBNP-induced AHbO2+MbO2 (% TLS) at rest versus the LBNP-induced decrease in ΔHbO2+MbO2 during handgrip. Group differences in ΔHbO2+MbO2 were assessed using a Student's t test. The drug treatment effect on LBNP-induced Δ·HbO2+MbO2 (% TLS) at rest versus the LBNP-induced decrease in ΔHbO2+MbO2 during handgrip was compared to baseline for each patient using a Student's t test. The group difference in the % Δ in brachial artery blood flow was assessed using a student's t test. The drug treatment effect on the % Δ in brachial artery blood flow was compared using a student's t test. All analyses were performed in SigmaPlot. Data are expressed as mean±SEM, unless otherwise specified.

Results (Analysis Using all 12 Boys with DMD and 10 Healthy Controls)

We studied 12 boys with DMD and 10 age-matched healthy male controls. Baseline characteristics and indices of disease severity of our DMD patients are shown in Table 3. All patients were ambulatory, although some of the boys used a wheelchair or scooter, especially to cover longer distances.

TABLE 3

| | | | Maximal | | | | |
|---|---|---|---|---|---|---|---|
| | | Age at | voluntary | | Creatine | Frequency of | |
| Patient | Exon | study | contraction | Dominant | Kinase | wheelchair/ | |
| No. | Mutation | (years) | (grip strength) | Hand | (IU/L) | scooter use | Medications |
| 3 | Duplication | 9 | 5 | Right | 8505 | Rarely | Deflazacort, |
| | 3-20 | | | | | | coQ10, hGH, |
| | | | | | | | Ca²⁺-carbonate, |
| | | | | | | | VD₃ |
| 4 | Deletion | 9 | 6 | Right | 6672 | Rarely | Delazacort, |
| | 38-43 | | | | | | Ca²⁺-carbonate, |
| | | | | | | | coQ10, MV, |
| | | | | | | | VD₃ |
| 5 | Deletion | 10 | 6 | Right | 10038 | Never | Delazacort, |
| | 46-52 | | | | | | Pepcid, |
| | | | | | | | Ca²⁺-carbonate |
| 6 | Duplication | 12 | 9 | Right | 1557 | Frequently | Prednisone, |
| | 3-44 | | | | | | Lisinopril, |
| | | | | | | | Ca²⁺-carbonate |
| 7 | Duplication | 12 | 10 | Left | 2246 | Frequently | Prednisone, |
| | 3-44 | | | | | | Lisinopril, |
| | | | | | | | Ca²⁺-carbonate |
| 10 | Deletion | 11 | 9 | Right | 6183 | Rarely | Deflazacort, |
| | 45 | | | | | | Ca²⁺-carbonate |
| 11 | Deletion | 11 | 9 | Right | 9493 | Rarely | Deflazacort, |
| | 43 | | | | | | losartan, hGH, |
| | | | | | | | protandim, |
| | | | | | | | Ca²⁺-carbonate, |
| | | | | | | | potassium, |
| | | | | | | | pamidronate, |
| 12 | Deletion | 8 | 10 | Right | 12659 | Rarely | Deflazacort, |
| | 36 | | | | | | lisinopril, |
| | | | | | | | VD₃, coQ10, |
| | | | | | | | creatine |
| | | | | | | | monohydrate, |
| | | | | | | | glutamine, MV |
| 13 | Deletion | 13 | 12 | Right | 2422 | Rarely | Deflazacort, |
| | 3-6 | | | | | | lisonopril, |
| | | | | | | | singulair, |
| | | | | | | | prevacid, MV |
| 14 | Deletion | 8 | 5 | Right | 6828 | Never | Prednisone, |
| | 45 | | | | | | prevacid |
| 15 | Deletion | 8 | 5 | Right | 11474 | Never | Prednisone, |
| | 34-35 | | | | | | CoQ10, |
| | | | | | | | Creatine, |
| | | | | | | | L-Glutamine, |
| | | | | | | | Fish Oil, VD₃ |
| 16 | Insertion | 8 | 3 | Right | 16403 | Frequently | Prednisone, |
| | 37 | | | | | | Lisinopril, |
| | | | | | | | coQ10, VD₃, |
| | | | | | | | Fluoxetine, |
| | | | | | | | rantitide |

As shown in Table 4, patients and controls were well matched for age, body mass index (BMI), blood pressure, and left ventricular ejection fraction. Resting heart rate was significantly higher in the boys with DMD (P<0.05), and as expected, grip strength was much lower in the boys with DMD compared to the controls (P<0.05).

TABLE 4
Baseline characteristics of patients with Duchenne muscular dystrophy compared to healthy controls

| | | Age | BMI | MVC | SBP | DBP | HR | LVEF |
|---|---|---|---|---|---|---|---|---|
| Group | N | (years) | (kg · m²) | (kg) | (mmHg) | mmHg) | (beats · min⁻¹) | (%) |
| Patients with DMD | 12 | 10 ± 1 | 28 ± 3 | 7 ± 1* | 105 ± 3 | 57 ± 3 | 91 ± 4* | 63 ± 2 |
| Healthy Controls | 10 | 11 ± 1 | 27 ± 2 | 19 ± 2 | 105 ± 3 | 56 ± 2 | 76 ± 2 | 64 ± 1 |

Mean ± SE.
*indicates significantly different from healthy control (P < 0.05)

Pharmakokinetic Studies and Safety Data

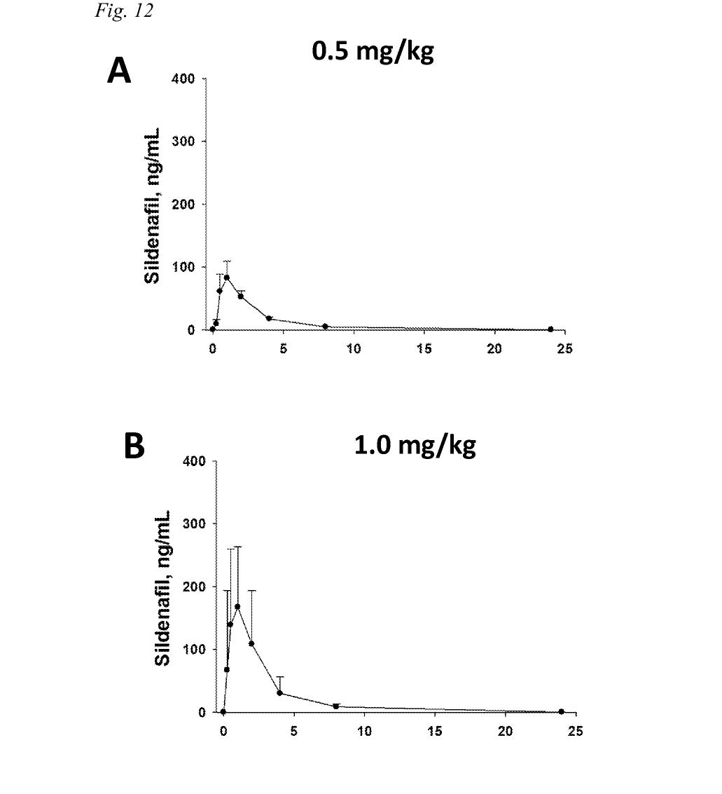

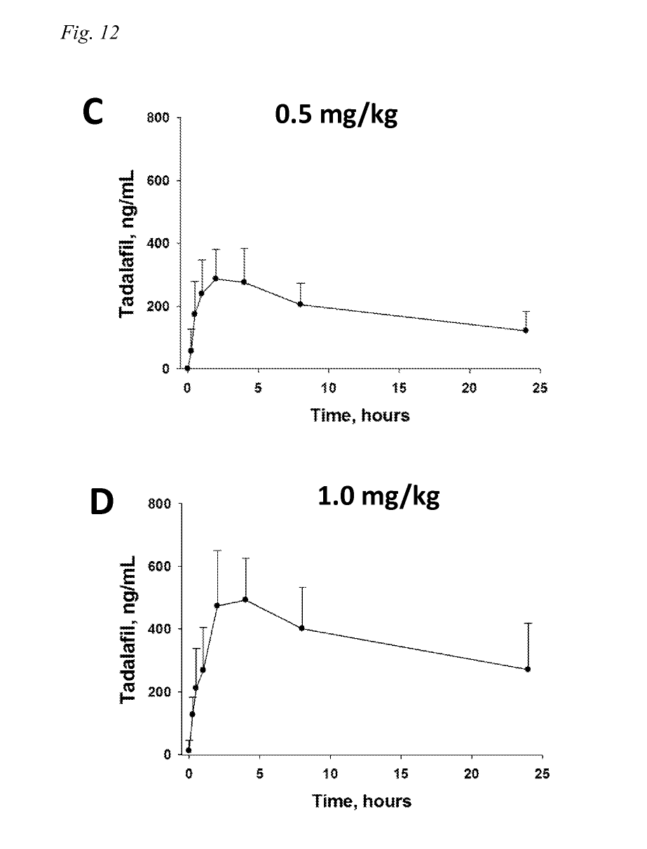

Nine patients with DMD participated in our pharmacokinetic studies assessing the time course of drug blood levels after two different oral doses (0.5 or 1.0 mg/kg) of sildenafil or tadalafil. One patient did not participate in the Sildenafil trial, and one patient developed an erection lasting longer than 4 hours following 0.5 mg/kg of tadalafil and was not escalated to the higher dose. The average group response curves are presented in FIG. 12. Peak sildenafil blood levels were achieved one hour after oral administration, increasing in a dose dependent manner: 0.5 mg/kg: 82.5±26.6 ng/mL; 1.0 mg/kg: 167.5±34.1 ng/mL. Likewise, tadalafil blood levels also responded in a dose-dependent manner, reaching a peak blood level between 2 and 4 hours after oral administration: 0.5 mg/kg: 286.31.3 ng/mL; 1.0 mg/kg: 492.5±47.3 ng/mL. As expected, tadalafil remained elevated in the blood for up to 24 hours, whereas sildenafil had returned close to baseline levels 4 hours after ingestion.

Facial flushing was extremely common with oral PDE5 inhibition, lasting several hours after treatment. Resting blood pressure was unaffected by either PDE5 inhibitor. As indicated above, one patient developed an erection lasting longer than 4 hours on the low dose of tadalafil, and was therefore not escalated to the higher dose. A second boy experienced a prolonged erection lasting 3 hours on the low dose of tadalafil, and 4 hours with the higher dose of tadalafil.

Functional Sympatholysis is Impaired in DMD and Restored by PDE5 Inhibition

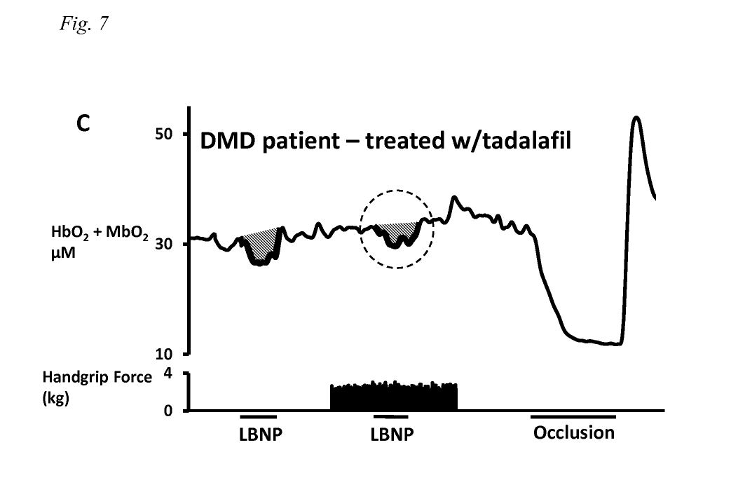

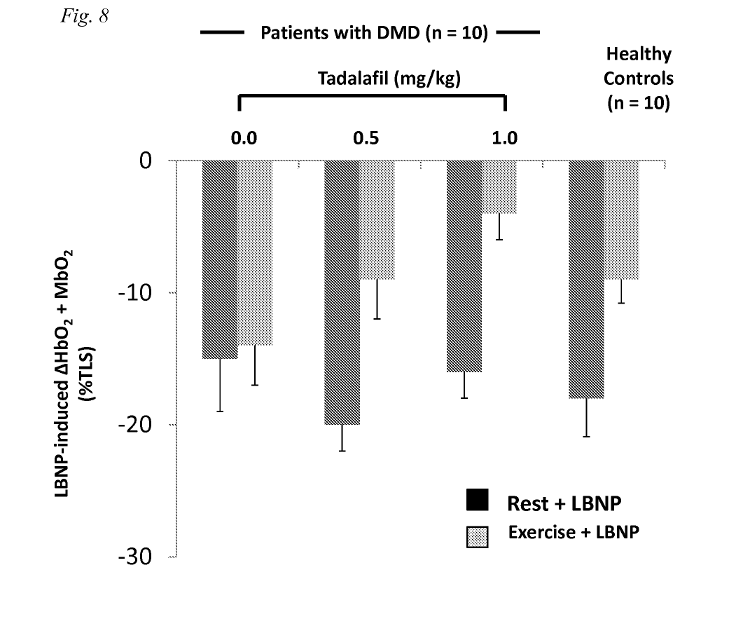

Functional sympatholysis—exercise induced attenuation of reflex sympathetic vasoconstriction—was assessed by applying the same reflex stimulus (lower body negative pressure) to the subjects forearm muscles at rest and during mild rhythmic handgrip exercise (FIG. 7). Sympathetic constriction of skeletal muscle microvessels was measured as the decrease in forearm muscle oxygenation by NIR spectroscopy. NIR spectroscopy was not possible in two DMD patients, due to severe movement artifact, thus data is reported in 10 subjects only. In resting forearm muscle, LBNP evoked comparable decreases in forearm muscle oxygenation (HbO₂+MBO₂) in DMD patients and healthy controls (FIG. 8). When LBNP was superimposed on handgrip in healthy controls, the reflex decrease in muscle oxygenation was attenuated by 54±8% (AHbO₂+MbO₂: −19±3% at rest versus −9±2% during handgrip, P<0.01), indicating functional sympatholysis. In contrast, no such attenuation was observed in patients with DMD (AHbO2+MbO2: −15±4% at rest versus −14±3% during handgrip), indicating impaired sympatholysis and thus functional muscle ischemia (FIG. 8). Of note, our results remain independent of prophylactic cardiac treatment with angiotensin converting enzyme inhibitors (namely Lisinopril) or angiotensin receptor blockers (namely Losartan, FIG. 12).

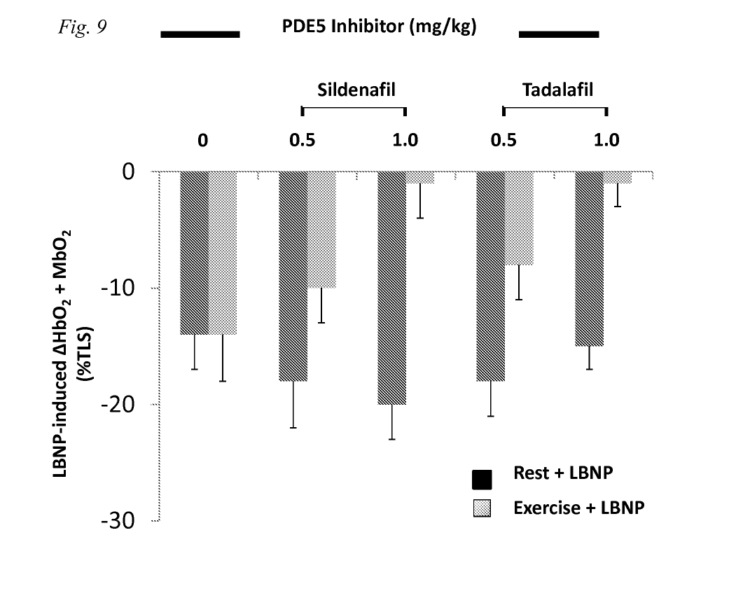

Next, we tested the ability of two different PDE5 inhibitors, sildenafil and tadalafil, to rescue functional sympatholysis in DMD. As shown in FIG. 8 and FIG. 9, tadalafil treatment restored functional sympatholysis in DMD patients in a dose-dependent manner. When LBNP was superimposed on handgrip exercise after 0.5 mg/kg tadalafil treatment, the reflex decrease in muscle oxygenation was attenuated by 57±16% (AHbO₂+MbO₂: −20±2% at rest versus −9±3% during handgrip, P<0.01). Furthermore, increasing the dose of tadalafil to 1.0 mg/kg caused even greater attenuation of the LBNP-induced decreases in muscle oxygenation: 80±11% (AHbO₂+MbO₂: −16±2% at rest versus −4±2% during handgrip, P<0.01). In a subset of patients, we also confirmed these results using a different PDE5 inhibitor (Sildenafil, FIG. 9): AHbO₂+MbO₂ 0.5 mg/kg: −18±4% at rest versus −10±3% during handgrip, P<0.05; 1.0 mg/kg: −20±3% at rest versus −1±3% during handgrip, P<0.01.

Post-Exercise Hyperemia is Blunted in DMD and Restored with PDE5 Inhibition

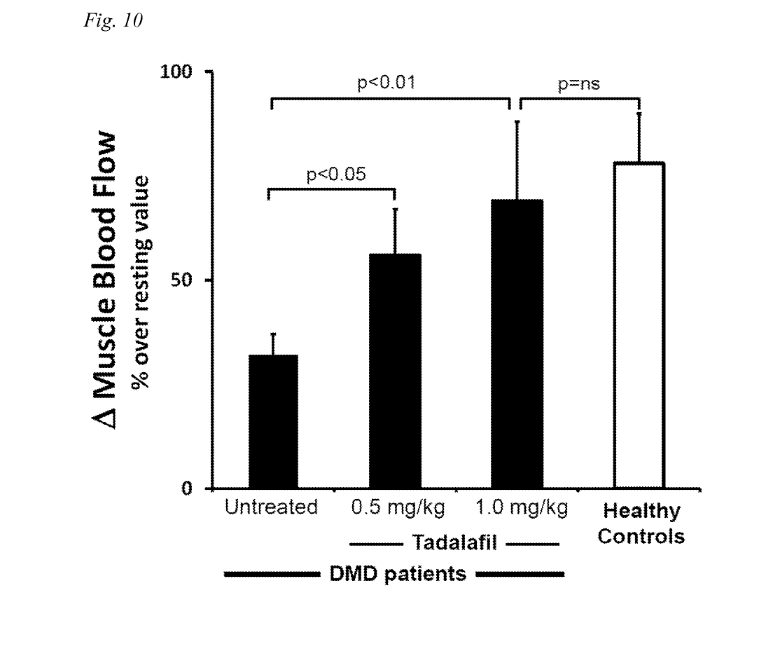

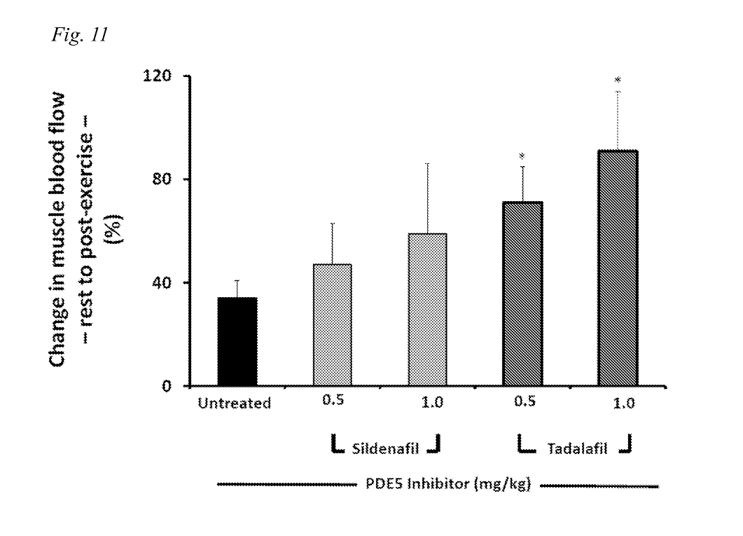

Post-exercise hyperemia—exercise induced increase in skeletal muscle blood flow—was assessed as the change in muscle blood flow from rest to immediately post-exercise, using pulsed-Doppler ultrasound of the brachial artery. As illustrated in FIG. 10, handgrip exercise caused a 71±13% increase in skeletal muscle blood flow in healthy controls. In contrast, in boys with DMD, handgrip exercise caused a much smaller increase in skeletal muscle blood flow (28±6%). PDE5 inhibition with either tadalafil or sildenafil restored post-exercise hyperemia and vascular conductance in patients with DMD (FIGS. 10 and 11). No differences in blood pressure, heart rate, or baseline blood flow were observed in response to either PDE5 inhibitor. Furthermore, no changes in brachial artery diameter were found with mild handgrip exercise. Thus, vascular conductance shared the same response as brachial artery blood flow (Table 5).

TABLE 5
Forearm blood flow and hemodynamics at rest and following rhythmic handgrip
exercise (20% maximal voluntary contraction) in patients with Duchenne muscular
dystrophy and age-matched healthy controls. Data reported as mean ± SE.

| | Duchenne Muscular Dystrophy | | | | | |
|---|---|---|---|---|---|---|
| | Baseline | | Tadalafil 0.5 mg/kg | | Tadalafil 1.0 mg/kg | |
| | | Post- | | Post- | | Post- |
| | Rest | exercise | Rest | exercise | Rest | exercise |
| Heart rate | 89 ± 3 | 91 ± 3 | 91 ± 3 | 94 ± 3 | 91 ± 3 | 92 ± 4 |
| MAP, mmHg | 75 ± 2 | 75 ± 2 | 74 ± 3 | 72 ± 2 | 71 ± 2 | 71 ± 3 |
| BA diameter, cm | 0.32 ± 0.02 | 0.33 ± 0.02 | 0.34 ± 0.02 | 0.34 ± 0.02 | 0.34 ± 0.02 | 0.34 ± 0.02 |
| BA velocity | 24 ± 3 | 31 ± 3 | 26 ± 4 | 38 ± 5 | 21 ± 3 | 35 ± 4 |
| BA blood flow, | 122 ± 15 | 154 ± 17 | 118 ± 15 | 184 ± 24 | 104 ± 14 | 170 ± 17 |
| ml/min | | | | | | |
| BA VC, | 1.7 ± 0.2 | 2.1 ± 0.2 | 1.7 ± 0.3 | 2.6 ± 0.4 | 1.5 ± 0.3 | 2.5 ± 0.3 |
| ml/min/mmHg | | | | | | |
| | | | Duchenne Muscular Dystrophy | | | |
| | Healthy Control | | Sildenafil 0.5 mg/kg | | Sildenafil 1.0 mg/kg | |
| | | Post- | | Post- | | Post- |
| | Rest | exercise | Rest | exercise | Rest | exercise |
| Heart rate | 76 ± 3 | 81 ± 3 | 84 ± 5 | 88 ± 6 | 92 ± 5 | 93 ± 5 |
| MAP, mmHg | 70 ± 1 | 73 ± 2 | 77 ± 6 | 75 ± 2 | 74 ± 2 | 74 ± 2 |
| BA diameter, cm | 0.33 ± 0.01 | 0.32 ± 0.01 | 0.32 ± 0.02 | 0.32 ± 0.02 | 0.33 ± 0.02 | 0.33 ± 0.01 |
| BA velocity | 19 ± 2 | 33 ± 3 | 24 ± 4 | 32 ± 3 | 27 ± 5 | 37 ± 3 |
| BA blood flow, | 103 ± 19 | 170 ± 30 | 125 ± 19 | 172 ± 18 | 137 ± 21 | 196 ± 22 |
| ml/min | | | | | | |
| BA VC, | 1.5 ± 0.3 | 2.4 ± 0.5 | 1.7 ± 0.3 | 2.3 ± 0.3 | 1.9 ± 0.3 | 2.7 ± 0.3 |
| ml/min/mmHg | | | | | | |

Results—(Analysis of 10 of the 12 Boys with DMD and 10 Healthy Controls)

Data are presented for 10 boys with DMD and 10 healthy controls; due to movement artifacts, data analysis in 2 boys with DMD was removed. Baseline characteristics and indices of disease severity of these 10 patients are shown in Table 6. All patients were ambulatory, although some often used a wheelchair or scooter. All DMD patients were receiving background therapy with glucocorticoids (either deflazacort or prednisone); five of the 10 patients also were receiving prophylactic cardiac medication (either lisinopril or losartan).

TABLE 6
Baseline characteristics of patients with Duchenne muscular dystrophy

| | | | Maximal | | | | |
|---|---|---|---|---|---|---|---|
| | | Age at | voluntary | | Creatine | Frequency of | |
| Patient | Exon | study | contraction | Dominant | Kinase | wheelchair/ | |
| No. | Mutation | (years) | (grip strength) | Hand | (IU/L) | scooter use | Medications |
| 1 | Deletion | 9 | 6 | Right | 6672 | Rarely | Delazacort, |
| | 38-43 | | | | | | Ca²⁺-carbonate, |
| | | | | | | | coQ10, MV, VD₃ |
| 2 | Deletion | 10 | 6 | Right | 10038 | Never | Delazacort, |
| | 46-52 | | | | | | Pepcid, |
| | | | | | | | Ca²⁺-carbonate |
| 3 | Duplication | 12 | 9 | Right | 1557 | Frequently | Prednisone, |
| | 3-44 | | | | | | Lisinopril, |
| | | | | | | | Ca²⁺-carbonate |
| 4 | Duplication | 12 | 10 | Left | 2246 | Frequently | Prednisone, |
| | 3-44 | | | | | | Lisinopril, |
| | | | | | | | Ca²⁺-carbonate |
| 5 | Deletion | 11 | 9 | Right | 6183 | Rarely | Defiazacort, |
| | 45 | | | | | | Ca²⁺-carbonate |
| 6 | Deletion | 11 | 9 | Right | 9493 | Rarely | Defiazacort, |
| | 43 | | | | | | losartan, hGH, |
| | | | | | | | protandim, |
| | | | | | | | Ca²⁺-carbonate, |
| | | | | | | | potassium, |
| | | | | | | | pamidronate, |
| 7 | Deletion | 8 | 10 | Right | 12659 | Rarely | Defiazacort, |
| | 36 | | | | | | lisinopril, VD₃, |
| | | | | | | | coQ10, creatine |
| | | | | | | | monohydrate, |
| | | | | | | | glutamine, MV |
| 8 | Deletion | 13 | 12 | Right | 2422 | Rarely | Defiazacort, |
| | 3-6 | | | | | | lisonopril, |
| | | | | | | | singulair, |
| | | | | | | | prevacid, MV |
| 9 | Deletion | 8 | 5 | Right | 6828 | Never | Prednisone, |
| | 45 | | | | | | prevacid |
| 10 | Deletion | 8 | 5 | Right | 11474 | Never | Prednisone, |
| | 34-35 | | | | | | CoQ10, Creatine, |
| | | | | | | | L-Glutamine, |
| | | | | | | | Fish Oil, VD₃ |

coQ10, coenzyme Q₁₀; hGH, human growth hormone; Ca²⁺-carbonate, calcium carbonate; VD₃, vitamin D₃ (cholecalciferol); MV, multivitamin.

Patients and controls were well matched for age, body mass index (BMI), blood pressure, and left ventricular ejection fraction (Table 7). As expected, resting heart rate was higher in the boys with DMD (P<0.05 vs. healthy controls) and maximal voluntary contraction (MVC, i.e., grip strength) was much lower (P<0.05).

TABLE 7
Baseline characteristics of patients with Duchenne muscular dystrophy compared to healthy controls

| | | Age | BMI | MVC | SBP | DBP | HR | LVEF |
|---|---|---|---|---|---|---|---|---|
| Group | N | (years) | (kg · m²) | (kg) | (mmHg) | mmHg) | (beats · min⁻¹) | (%) |
| Patients with DMD | 10 | 10 ± 1 | 28 ± 3 | 8 ± 1* | 106 ± 3 | 57 ± 3 | 91 ± 5* | 63 ± 2 |
| Healthy Controls | 10 | 11 ± 1 | 27 ± 2 | 19 ± 2 | 105 ± 3 | 56 ± 2 | 76 ± 2 | 64 ± 1 |

Data reported as mean ± SE.
*indicates significantly different from healthy control (P < 0.05).
BMI, body mass index; MVC, maximal voluntary contraction; SBP, systolic blood pressure; DBP, diastolic blood pressure; HR, heart rate; LVEF, left ventricular ejection fraction.

Functional Sympatholysis is Impaired in DMD and Restored by PDE5 Inhibition

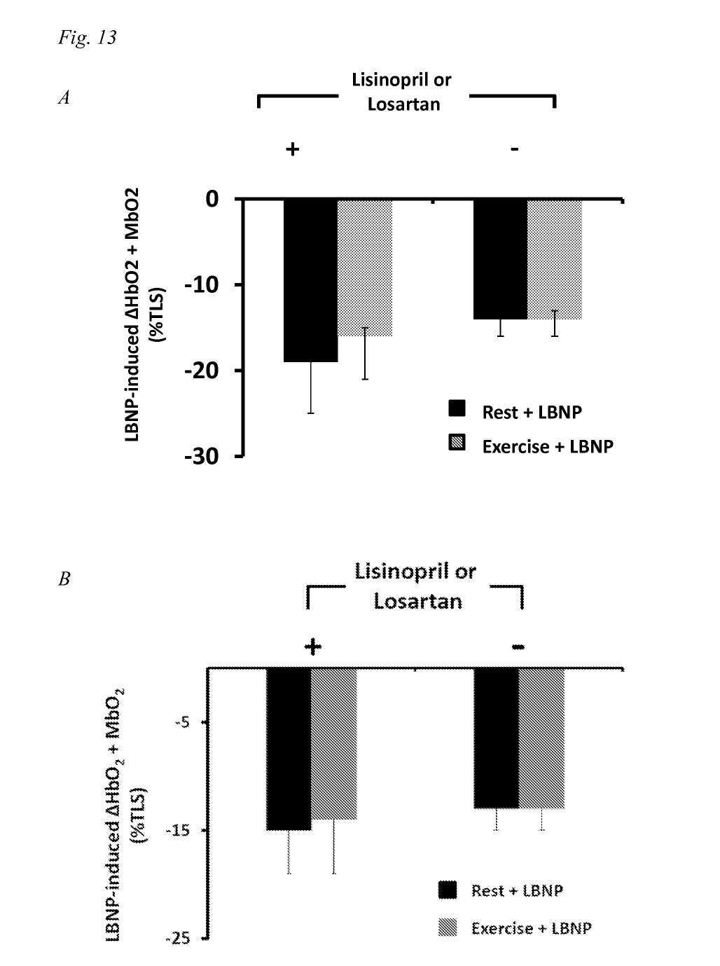

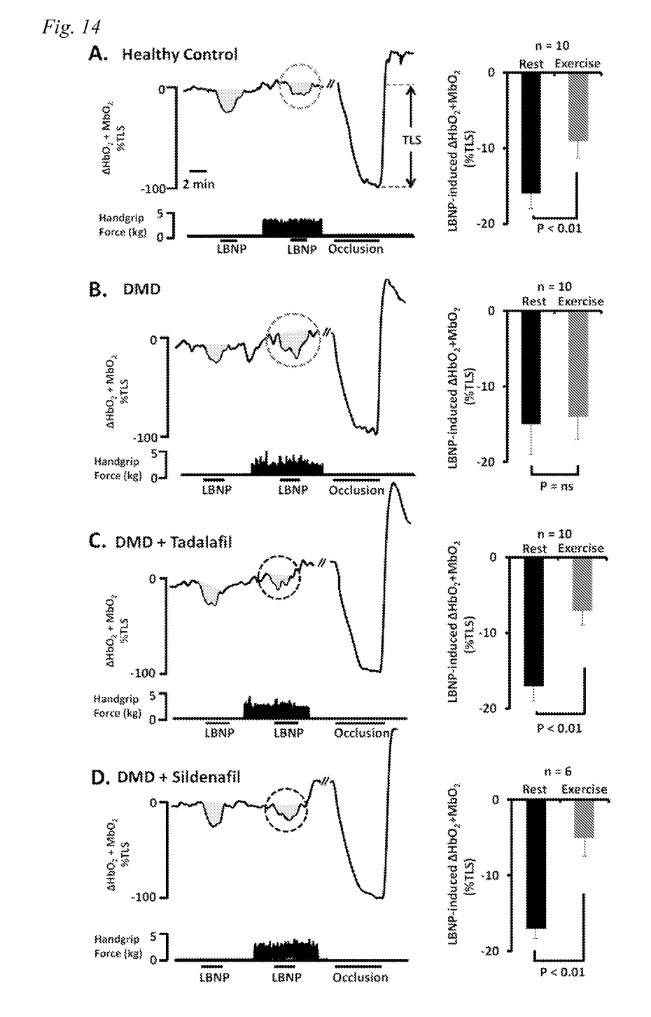

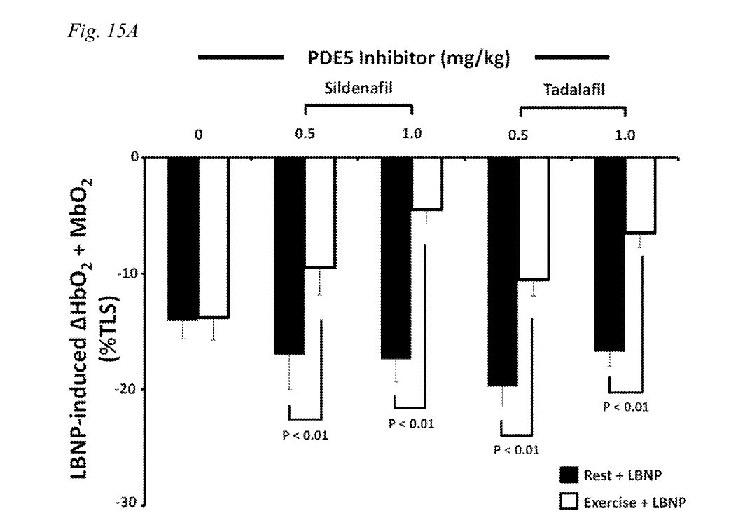

In resting forearm muscle, LBNP evoked comparable decreases in forearm muscle oxygenation (HbO₂+MBO₂) in patients and controls (FIG. 14). When LBNP was superimposed on handgrip in healthy controls, the reflex decrease in muscle oxygenation was attenuated by 54±8% (AHbO₂+MbO₂: −18±3% at rest versus −9±2% during handgrip, P<0.01), indicating functional sympatholysis. In contrast, no such attenuation was observed in the patients with DMD (AHbO2+MbO2: −14±2% at rest versus −13±2% during handgrip), indicating functional muscle ischemia (FIG. 14). In boys with DMD, sympatholysis was similarly impaired in those treated (n=5) or not treated (n=5) with prophylactic cardiac medication (FIG. 13B).

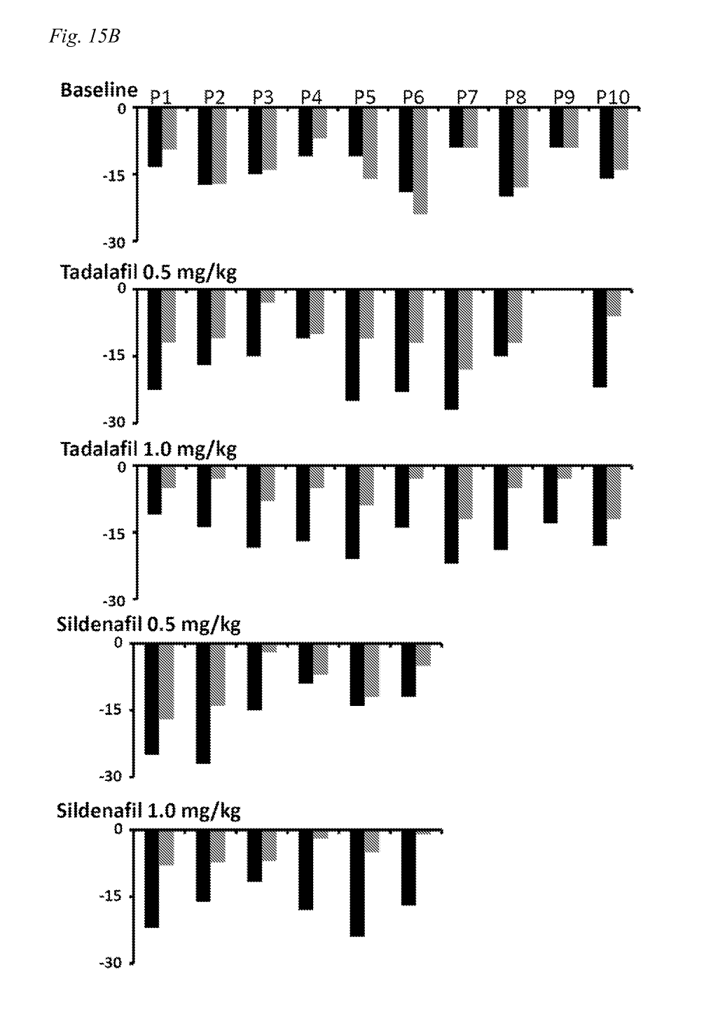

Tadalafil restored functional sympatholysis in boys with DMD (FIG. 14) in a dose-dependent manner. With the 0.5 mg/kg dose of tadalafil, the reflex decrease in muscle oxygenation was attenuated by 45±8% (AHbO₂+MbO₂: −20±2% at rest versus −11±1% during handgrip, P<0.01), a response that is indistinguishable from normal (P=ns vs. healthy controls). The 1.0 mg/kg dose caused a super-normal 63±5% attenuation: (AHbO₂+MbO₂: −17±1% at rest versus −7±1% during handgrip, P<0.01). In a subset of patients (n=6), we confirmed these results with sildenafil (FIGS. 14 and 15A). Individual data are illustrated in supplemental FIG. 15B.

Exercise-Induced Hyperemia is Blunted in Boys with DMD and Restored by Tadalafil

Handgrip exercise increased brachial artery blood flow by 78±12% over baseline in healthy controls but only by 32±5% in boys with DMD (p<0.05, FIG. 10). Remarkably, in DMD, tadalafil restored exercise-induced hyperemia in a dose-dependent manner (Table 5). A similar tendency was seen with sildenafil but the treatment effect was not statistically significant (FIG. 11).

Pharmakokinetic Data

Nine boys with DMD completed the pharmacokinetic study. The average group response curves are presented in FIG. 12. Blood levels peaked approximately one hour after oral sildenafil administration with an elimination half-life of 2.4±0.09 hours for the 0.5 mg/kg dose, and 3.2±0.5 hours for the 1.0 mg/kg dose. In contrast, blood levels peaked 2-4 hours after oral tadalafil administration, with an elimination half-life of 24.9±3.1 hours for the 0.5 mg/kg dose, and 39.5±6.6 hours for the 1.0 mg/kg dose.

Safety Data

Facial flushing occurred in all boys with both doses of either PDE5 inhibitor. Blood pressure was unaffected by either drug. One patient developed a penile erection lasting 6 hours after low-dose of tadalafil, and was not escalated to the higher dose. A second boy experienced erection lasting 3 hours after low-dose of tadalafil, and 4 hours after high-dose tadalafil. In both cases, erections were non-painful and resolved spontaneously.

### REFERENCES

1. K. Bushby, R. Finkel, D. J. Birnkrant, L. E. Case, P. R. Clemens, L. Cripe, A. Kaul, K. Kinnett, C. McDonald, S. Pandya, J. Poysky, F. Shapiro, J. Tomersko, C. Constantin, Diagnosis and management of Duchenne muscular dystrophy, part 1: diagnosis, and pharmacological and psychosocial management, Lancet Neurol., 9, 77-93 (2010).
2. K. Bushby, R. Finkel, D. J. Birnkrant, L. E. Case, P. R. Clemens, L. Cripe, A. Kaul, K. Kinnett, C. McDonald, S. Pandya, J. Poysky, F. Shapiro, J. Tomersko, C. Constantin, Diagnosis and management of Duchenne muscular dystrophy, part 2: implementation of multidisciplinary care, Lancet Neurol., 9, 177-189 (2010).
3. A. E. Emery, The muscular dystrophies, Lancet, 359, 687-695 (2002).
4. M. Koenig, A. H. Beggs, M. Moyer, S. Scherpf, K. Heindrich, T. Bettecken, G. Meng, C. R. Muller, M. Lindlof, H. Kaariainen, The molecular basis for Duchenne versus Becker muscular dystrophy: correlation of severity with type of deletion, Am. J. Hum. Genet., 45, 498-506 (1989).
5. A. P. Monaco, C. J. Bertelson, S. Liechti-Gallati, H. Moser, L. M. Kunkel, An explanation for the phenotypic differences between patients bearing partial deletions of the DMD locus, Genomics, 2, 90-95 (1988).
6. D. Duan, Duchenne muscular dystrophy gene therapy: Lost in translation?, Res. Rep. Biol., 2011, 31-42 (2011).
7. D. J. Blake, A. Weir, S. E. Newey, K. E. Davies, Function and genetics of dystrophin and dystrophin-related proteins in muscle, Physiol Rev., 82, 291-329 (2002).
8. B. J. Petrof, J. B. Shrager, H. H. Stedman, A. M. Kelly, H. L. Sweeney, Dystrophin protects the sarcolemma from stresses developed during muscle contraction, Proc. Natl. Acad. Sci. USA, 90, 3710-3714 (1993).
9. J. E. Brenman, D. S. Chao, H. Xia, K. Aldape, D. S. Bredt, Nitric oxide synthase complexed with dystrophin and absent from skeletal muscle sarcolemma in Duchenne muscular dystrophy, Cell, 82, 743-752 (1995).
10. W. J. Chang, S. T. Iannaccone, K. S. Lau, B. S. Masters, T. J. McCabe, K. McMillan, R. C. Padre, M. J. Spencer, J. G. Tidball, J. T. Stull, Neuronal nitric oxide synthase and dystrophindeficient muscular dystrophy, Proc. Natl. Acad. Sci. USA, 93, 9142-9147 (1996).
11. Y. Lai, G. D. Thomas, Y. Yue, H. T. Yang, D. Li, C. Long, L. Judge, B. Bostick, J. S. Chamberlain, R. L. Terjung, D. Duan, Dystrophins carrying spectrin-like repeats 16 and 17 anchor nNOS to the sarcolemma and enhance exercise performance in a mouse model of muscular dystrophy, J. Clin. Invest., 119, 624-635 (2009).
12. B. Chavoshan, M. Sander, T. E. Sybert, J. Hansen, R. G. Victor, G. D. Thomas, Nitric oxidedependent modulation of sympathetic neural control of oxygenation in exercising human skeletal muscle, J. Physiol., 540, 377-386 (2002).
13. J. Hansen, G. D. Thomas, S. A. Harris, W. J. Parsons, R. G. Victor, Differential sympathetic neural control of oxygenation in resting and exercising human skeletal muscle, J. Clin. Invest., 98, 584-596 (1996).
14. M. Sander, B. Chavoshan, S. A. Harris, S. T. Iannaccone, J. T. Stull, G. D. Thomas, R. G. Victor, Functional muscle ischemia in neuronal nitric oxide synthase-deficient skeletal muscle of children with Duchenne muscular dystrophy, Proc. Natl. Acad. Sci. USA, 97, 13818-13823 (2000).
15. G. D. Thomas, J. Hansen, R. G. Victor, Inhibition of alpha 2-adrenergic vasoconstriction during contraction of glycolytic, not oxidative, rat hindlimb muscle, Am. J. Physiol., 266, H920-H929 (1994).
16. G. D. Thomas, M. Sander, K. S. Lau, P. L. Huang, J. T. Stull, R. G. Victor, Impaired metabolic modulation of alpha-adrenergic vasoconstriction in dystrophin-deficient skeletal muscle, Proc. Natl. Acad. Sci. USA, 95, 15090-15095 (1998).
17. G. D. Thomas, R. G. Victor, Nitric oxide mediates contraction-induced attenuation of sympathetic vasoconstriction in rat skeletal muscle, J. Physiol., 506 (Pt 3), 817-826 (1998).
18. G. D. Thomas, W. Zhang, R. G. Victor, Impaired modulation of sympathetic vasoconstriction in contracting skeletal muscle of rats with chronic myocardial infarctions: role of oxidative stress, Circ. Res., 88, 816-823 (2001).
19. G. D. Thomas, P. W. Shaul, I. S. Yuhanna, S. C. Froehner, M. E. Adams, Vasomodulation by skeletal muscle-derived nitric oxide requires alpha-syntrophin-mediated sarcolemmal localization of neuronal Nitric oxide synthase, Circ. Res., 92, 554-560 (2003).
20. W. Vongpatanasin, Z. Wang, D. Arbique, G. Arbique, B. Adams-Huet, J. H. Mithcell, R. G. Victor, G. D. Thomas, Functional sympatholysis is impaired in hypertensive humans, J. Physiol., 589, 1209-1220 (2011).
21. B. G. Wallin, R. G. Victor, A. L. Mark, Sympathetic outflow to resting muscles during static handgrip and postcontraction muscle ischemia, Am. J. Physiol., 256, H105-H110 (1989).
22. M. Wehling-Henricks, M. C. Jordan, K. P. Roos, B. Deng, J. G. Tidball, Cardiomyopathy in dystrophin-deficient hearts is prevented by expression of a neuronal nitric oxide synthase transgene in the myocardium, Hum. Mol. Genet., 14, 1921-1933 (2005).
23. M. Wehling, M. J. Spencer, J. G. Tidball, A nitric oxide synthase transgene ameliorates muscular dystrophy in mdx mice, J. Cell Biol., 155, 123-131 (2001).
24. E. R. Barton, L. Morris, M. Kawana, L. T. Bish, T. Toursel, Systemic administration of L-arginine benefits mdx skeletal muscle function, Muscle Nerve, 32, 751-760 (2005).
25. V. Voisin, C. Sebrie, S. Matecki, H. Yu, B. Gillet, M. Ramonatxo, M. Israel, S. De la Porte, L-arginine improves dystrophic phenotype in mdx mice, Neurobiol. Dis., 20, 123-130 (2005).
26. S. Brunelli, C. Sciorati, G. D'Antona, A. Innocenzi, D. Covarello, B. G. Galvez, C. Perrotta, A. Monopoli, F. Sanvito, R. Bottinelli, E. Ongini, G. Cossu, E. Clementi, Nitric oxide release combined with nonsteroidal antiinflammatory activity prevents muscular dystrophy pathology and enhances stem cell therapy, Proc. Natl. Acad. Sci. U.S.A, 104, 264-269 (2007).
27. C. Sciorati, D. Miglietta, R. Buono, V. Pisa, D. Cattaneo, E. Azzoni, S. Brunelli, E. Clementi, A dual acting compound releasing nitric oxide (NO) and ibuprofen, NCX 320, shows significant therapeutic effects in a mouse model of muscular dystrophy, Pharmacol. Res., 64, 210-217 (2011).
28. A. Asai, N. Sahani, M. Kaneki, Y. Ouchi, J. A. Martyn, S. E. Yasuhara, Primary role of functional ischemia, quantitative evidence for the two-hit mechanism, and phosphodiesterase-5 inhibitor therapy in mouse muscular dystrophy, PLoS. One., 2, e806-(2007).
29. Y. M. Kobayashi, E. P. Rader, R. W. Crawford, N. K. Iyengar, D. R. Thedens, J. A. Faulkner, S. V. Parikh, R. M. Weiss, J. S. Chamberlain, S. A. Moore, K. P. Campbell, Sarcolemma-localized nNOS is required to maintain activity after mild exercise, Nature, 456, 511-515 (2008).
30. C. M. Adamo, D. F. Dai, J. M. Percival, E. Minami, M. S. Willis, E. Patrucco, S. C. Froehner, J. A. Beavo, Sildenafil reverses cardiac dysfunction in the mdx mouse model of Duchenne muscular dystrophy, Proc. Natl. Acad. Sci. USA, 107, 19079-19083 (2010).
31. M. Khairallah, R. J. Khairallah, M. E. Young, B. G. Allen, M. A. Gillis, G. Danialou, C. F. Deschepper, B. J. Petrof, R. C. Des, Sildenafil and cardiomyocyte-specific cGMP signaling prevent cardiomyopathic changes associated with dystrophin deficiency, Proc. Natl. Acad. Sci. USA, 105, 7028-7033 (2008).
32. G. Kawahara, J. A. Karpf, J. A. Myers, M. S. Alexander, J. R. Guyon, L. M. Kunkel, Drug screening in a zebrafish model of Duchenne muscular dystrophy, Proc. Natl. Acad. Sci. USA, 108, 5331-5336 (2011).
33. D. S. Chao, J. R. Gorospe, J. E. Brenman, J. A. Rafael, M. F. Peters, S. C. Froehner, E. P. Hoffman, J. S. Chamberlain, D. S. Bredt, Selective loss of sarcolemmal nitric oxide synthase in Becker muscular dystrophy, J. Exp. Med., 184, 609-618 (1996).
34. S. Torelli, S. C. Brown, C. Jimenez-Mallebrera, L. Feng, F. Muntoni, C. A. Sewry, Absence of neuronal nitric oxide synthase (nNOS) as a pathological marker for the diagnosis of Becker muscular dystrophy with rod domain deletions, Neuropathol. Appl. Neurobiol., 30, 540-545 (2004).
35. K. E. Wells, S. Torelli, Q. Lu, S. C. Brown, T. Partridge, F. Muntoni, D. J. Wells, Relocalization of neuronal nitric oxide synthase (nNOS) as a marker for complete restoration of the dystrophin associated protein complex in skeletal muscle, Neuromuscul. Disord., 13, 21-31 (2003).
36. A. Romfh, E. M. McNally, Cardiac assessment in duchenne and becker muscular dystrophies, Curr. Heart Fail. Rep., 7, 212-218 (2010).
37. W. Zhao, S. A. Swanson, J. Ye, X. Li, J. M. Shelton, W. Zhang, G. D. Thomas, Reactive oxygen species impair sympathetic vasoregulation in skeletal muscle in angiotensin II dependent hypertension, Hypertension, 48, 637-643 (2006).
38. P. Bagher, D. Duan, S. S. Segal, Evidence for impaired neurovascular transmission in a murine model of Duchenne muscular dystrophy, J. Appl. Physiol, 110, 601-609 (2011).
39. J. H. Lombard, Microcirculation in a mouse model of Duchenne muscular dystrophy: another blow to the vascular hypothesis?, J. Appl. Physiol, 110, 587-588 (2011).
40. C. Gentil, F. Leturcq, Y. R. Ben, J. C. Kaplan, P. Laforet, I. Penisson-Besnier, C. Espil-Taris, T. Voit, L. Garcia, F. Pietri-Rouxel, Variable phenotype of del45-55 Becker patients correlated with nNOSmu mislocalization and RYR1 hypernitrosylation, Hum. Mol. Genet., 21, 3449-3460 (2012).
41. V. Palit, I. Eardley, An update on new oral PDE5 inhibitors for the treatment of erectile dysfunction, Nat. Rev. Urol., 7, 603-609 (2010).
42. G. Ravipati, J. A. McClung, W. S. Aronow, S. J. Peterson, W. H. Frishman, Type 5 phosphodiesterase inhibitors in the treatment of erectile dysfunction and cardiovascular disease, Cardiol. Rev., 15, 76-86 (2007).
43. R. W. Kaspar, H. D. Allen, W. C. Ray, C. E. Alvarez, J. T. Kissel, A. Pestronk, R. B. Weiss, K. M. Flanigan, J. R. Mendell, F. Montanaro, Analysis of dystrophin deletion mutations predicts age of cardiomyopathy onset in becker muscular dystrophy, Circ. Cardiovasc. Genet., 2, 544-551 (2009).
44. P. J. Fadel, I. M. Farias, K. M. Gallagher, Z. Wang, G. D. Thomas, Oxidative stress and enhanced sympathetic vasoconstriction in contracting muscles of nitrate-tolerant rats and humans, J. Physiol, 590, 395-407 (2012).
45. J. M. Percival, K. N. Anderson, P. Huang, M. E. Adams, S. C. Froehner, Golgi and sarcolemmal neuronal NOS differentially regulate contraction-induced fatigue and vasoconstriction in exercising mouse skeletal muscle, J. Clin. Invest, 120, 816-826 (2010).
46. K. Anthony, S. Cirak, S. Torelli, G. Tasca, L. Feng, V. Arechavala-Gomeza, A. Armaroli, M. Guglieri, C. S. Straathof, J. J. Verschuuren, A. Aartsma-Rus, P. Helderman-van den Enden, K. Bushby, V. Straub, C. Sewry, A. Ferlini, E. Ricci, J. E. Morgan, F. Muntoni, Dystrophin quantification and clinical correlations in Becker muscular dystrophy: implications for clinical trials, Brain, 134, 3547-3559 (2011).
47. E. L. Finanger Hedderick, J. L. Simmers, A. Soleimani, E. Andres-Mateos, R. Marx, D. C. Files, L. King, T. O. Crawford, A. M. Corse, R. D. Cohn, Loss of sarcolemmal nNOS is common in acquired and inherited neuromuscular disorders, Neurology, 76, 960-967 (2011).
48. NIH RePORT, Planning a Multicenter Trial of PDE5A Inhibition for Duchenne Muscular Dystrophy, http://projectreporter.nih.gov/project_info_description.cfm?aid=8295579&icde=13915249&ddparam=&ddvalue=&ddsub=&cr=1&csb=default&cs=ASC, Last Accessed Sep. 26, 2012.
49. S. Cirak, V. Arechavala-Gomeza, M. Guglieri, L. Feng, S. Torelli, K. Anthony, S. Abbs, M. E. Garralda, J. Bourke, D. J. Wells, G. Dickson, M. J. Wood, S. D. Wilton, V. Straub, R. Kole, S. B. Shrewsbury, C. Sewry, J. E. Morgan, K. Bushby, F. Muntoni, Exon skipping and dystrophin restoration in patients with Duchenne muscular dystrophy after systemic phosphorodiamidate morpholino oligomer treatment: an open-label, phase 2, dose-escalation study, Lancet, 378, 595-605 (2011).
50. S. Cirak, L. Feng, K. Anthony, V. Arechavala-Gomeza, S. Torelli, C. Sewry, J. E. Morgan, F. Muntoni, Restoration of the Dystrophin-associated Glycoprotein Complex After Exon Skipping Therapy in Duchenne Muscular Dystrophy, Mol. Ther., 20, 462-467 (2012).
51. N. Suzuki, N. Motohashi, A. Uezumi, S. Fukada, T. Yoshimura, Y. Itoyama, M. Aoki, Y. Miyagoe-Suzuki, S. Takeda, NO production results in suspension-induced muscle atrophy through dislocation of neuronal NOS, J. Clin. Invest, 117, 2468-2476 (2007).
52. F. Zeng, Z. R. Ren, S. Z. Huang, M. Kalf, M. Mommersteeg, M. Smit, S. White, C. L. Jin, M. Xu, D. W. Zhou, J. B. Yan, M. J. Chen, B. R. van, S. Z. Huang, D. J. den, Y. T. Zeng, Y. Wu, Array-MLPA: comprehensive detection of deletions and duplications and its application to DMD patients, Hum. Mutat., 29, 190-197 (2008).
53. T. Lalic, R. H. Vossen, J. Coffa, J. P. Schouten, M. Guc-Scekic, D. Radivojevic, M. Djurisic, M. H. Breuning, S. J. White, J. T. den Dunnen, Deletion and duplication screening in the DMD gene using MLPA, Eur. J. Hum. Genet., 13, 1231-1234 (2005).
54. K. M. Flanigan, N. A. von, D. M. Dunn, J. Alder, J. R. Mendell, R. B. Weiss, Rapid direct sequence analysis of the dystrophin gene, Am. J. Hum. Genet., 72, 931-939 (2003).
55. E. Gratton, S. Fantini, M. A. Franceschini, G. Gratton, M. Fabiani, Measurements of scattering and absorption changes in muscle and brain, Philos. Trans. R. Soc. Lond B Biol. Sci., 352, 727-735 (1997).
56. F. F. Jobsis, Noninvasive, infrared monitoring of cerebral and myocardial oxygen sufficiency and circulatory parameters, Science, 198, 1264-1267 (1977).
57. R. G. Victor, D. R. Seals, A. L. Mark, Differential control of heart rate and sympathetic nerve activity during dynamic exercise. Insight from intraneural recordings in humans, J. Clin. Invest, 79, 508-516 (1987).
58. T. N. Jacobsen, B. J. Morgan, U. Scherrer, S. F. Vissing, R. A. Lange, N. Johnson, W. S. Ring, P. S. Rahko, P. Hanson, R. G. Victor, Relative contributions of cardiopulmonary and sinoaortic baroreflexes in causing sympathetic activation in the human skeletal muscle circulation during orthostatic stress, Circ. Res., 73, 367-378 (1993).
59. R. G. Victor, W. N. Leimbach, Jr., Effects of lower body negative pressure on sympathetic discharge to leg muscles in humans, J. Appl. Physiol, 63, 2558-2562 (1987).
60. S. F. Vissing, U. Scherrer, R. G. Victor, Relation between sympathetic outflow and vascular resistance in the calf during perturbations in central venous pressure. Evidence for cardiopulmonary afferent regulation of calf vascular resistance in humans, Circ. Res., 65, 1710-1717 (1989).

61. L. V. Anderson, K. Davison, Multiplex Western blotting system for the analysis of muscular dystrophy proteins, Am. J. Pathol., 154, 1017-1022 (1999).

### REFERENCES

(1B) Emery A E H. Population frequencies of inherited neuromuscular diseases—A world survey. Neuromuscular Disorders 1991; 1(1):19-29.
(2B) Thomas G D, Sander M, Lau K S, Huang P L, Stull J T, Victor R G. Impaired metabolic modulation of alpha-adrenergic vasoconstriction in dystrophin-deficient skeletal muscle. Proceedings of the National Academy of Sciences 1998 Dec. 8; 95(25):15090-5.
(3B) Thomas G D, Ye J, De Nardi C, Monopoli A, Ongini E, Victor R G. Treatment with a Nitric Oxide-Donating NSAID Alleviates Functional Muscle Ischemia in the Mouse Model of Duchenne Muscular Dystrophy. PLoS ONE 2012 Nov. 5; 7(11):e49350.
(4B) Martin E A, Barresi R, Byrne B J et al. Tadalafil Alleviates Muscle Ischemia in Patients with Becker Muscular Dystrophy. Science Translational Medicine 2012 Nov. 28; 4(162):162ra155.
(5B) Sander M, Chavoshan B, Harris S A et al. Functional muscle ischemia in neuronal nitric oxide synthase-deficient skeletal muscle of children with Duchenne muscular dystrophy. Proceedings of the National Academy of Sciences 2000 Dec. 5; 97(25):13818-23.
(6B) Asai A, Sahani N, Kaneki M, Ouchi Y, Martyn J A J, Yasuhara S E. Primary Role of Functional Ischemia, Quantitative Evidence for the Two-Hit Mechanism, and Phosphodiesterase-5 Inhibitor Therapy in Mouse Muscular Dystrophy. PLoS ONE 2007 Aug. 29; 2(8):e806.
(7B) Kobayashi Y M, Rader E P, Crawford R W et al. Sarcolemma-localized nNOS is required to maintain activity after mild exercise. Nature 2008 Nov. 27; 456(7221):511-5.
(8B) Herr M D, Hogeman C S, Koch D W, Krishnan A, Momen A, Leuenberger U A. A real-time device for converting Doppler ultrasound audio signals into fluid flow velocity. American Journal of Physiology—Heart and Circulatory Physiology 2010 May 1; 298(5):H1626-H1632.
(9B) Hansen J, Thomas G D, Harris S A, Parsons W J, Victor R G. Differential sympathetic neural control of oxygenation in resting and exercising human skeletal muscle. J Clin Invest 1996 Jul. 15; 98(2):584-96.
(10B) Victor R G, Seals D R, Mark A L. Differential control of heart rate and sympathetic nerve activity during dynamic exercise. Insight from intraneural recordings in humans. J Clin Invest 1987 Feb. 1; 79(2):508-16.
(11B) Thomas G D, Shaul P W, Yuhanna I S, Froehner S C, Adams M E. Vasomodulation by Skeletal Muscle-Derived Nitric Oxide Requires alpha-Syntrophin-Mediated Sarcolemmal Localization of Neuronal Nitric Oxide Synthase. Circ Res 2003 Mar. 21; 92(5):554-60.
(12B) Vongpatanasin W, Wang Z, Arbique D et al. Functional sympatholysis is impaired in hypertensive humans. The Journal of Physiology 2011 Mar. 1; 589(5):1209-20.
(13B) Zhao W, Swanson S A, Ye J et al. Reactive Oxygen Species Impair Sympathetic Vasoregulation in Skeletal Muscle in Angiotensin IIG [symbol] ôDependent Hypertension. Hypertension 2006 Oct. 1; 48(4):637-43.
(14B) Gao L, Wang W, Li Y L et al. Superoxide Mediates Sympathoexcitation in Heart Failure: Roles of Angiotensin II and NAD(P)H Oxidase. Circ Res 2004 Oct. 29; 95(9):937-44.
(15B) Chavoshan B, Sander M, Sybert T E, Hansen J, Victor R G, Thomas G D. Nitric oxide-dependent modulation of sympathetic neural control of oxygenation in exercising human skeletal muscle. The Journal of Physiology 2002 Apr. 1; 540(1):377-86.
(16B) Thomas G D, Hansen J, Victor R G Inhibition of alpha 2-adrenergic vasoconstriction during contraction of glycolytic, not oxidative, rat hindlimb muscle. American Journal of Physiology—Heart and Circulatory Physiology 1994 Mar. 1; 266(3):H920-H929.
(17B) Thomas G D, Victor R G. Nitric oxide mediates contraction-induced attenuation of sympathetic vasoconstriction in rat skeletal muscle. The Journal of Physiology 1998 Feb. 1; 506(3):817-26.
(18B) Thomas G D, Zhang W, Victor R G. Impaired Modulation of Sympathetic Vasoconstriction in Contracting Skeletal Muscle of Rats With Chronic Myocardial Infarctions: Role of Oxidative Stress. Circ Res 2001 Apr. 27; 88(8):816-23.
(19B) Brenman J E, Chao D S, Xia H, Aldape K, Bredt D S. Nitric oxide synthase complexed with dystrophin and absent from skeletal muscle sarcolemma in Duchenne muscular dystrophy. Cell 1995 Sep. 8; 82(5):743-52.
(20B) Chang W J, Iannaccone S T, Lau K S et al. Neuronal nitric oxide synthase and dystrophin-deficient muscular dystrophy. Proceedings of the National Academy of Sciences 1996 Aug. 20; 93(17):9142-7.
(21B) Lai Y, Thomas G D, Yue Y et al. Dystrophins carrying spectrin-like repeats 16 and 17 anchor nNOS to the sarcolemma and enhance exercise performance in a mouse model of muscular dystrophy. J Clin Invest 2009 Mar. 2; 119(3):624-35.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A method of treating muscular dystrophy, comprising:
providing a phosodiesterase 5A (PDE5A) inhibitor or a salt thereof; and
administering a quantity of a PDE5A inhibitor to a subject in need of treatment for muscular dystrophy.

2. The method of claim 1, wherein the muscular dystrophy is Becker muscular dystrophy (BMD).

3. The method of claim 1, wherein the muscular dystrophy is Duchenne muscular dystrophy (DMD).

4. A method of restoring functional sympatholysis, alleviating ischemic insult to dystrophin-deficient muscle membranes, reducing use-dependent muscle injury, or alleviating post-exercise hyperemia, comprising
providing a phosodiesterase 5A (PDE5A) inhibitor or a salt thereof; and
administering a quantity of a PDE5A inhibitor to a subject in need of restoring functional sympatholysis, alleviating ischemic insult to dystrophin-deficient muscle membranes, reducing use-dependent muscle injury, or alleviating post-exercise hyperemia.

5. (canceled)

6. (canceled)

7. (canceled)

8. The method of claim 1, wherein the PDE5A inhibitor is tadalafil or a salt thereof.

9. The method of claim 1, wherein the PDE5A inhibitor is sildenafil or a salt thereof.

10. The method of claim 1, wherein the PDE5A inhibitor is vardenafil or a salt thereof.

11. The method of claim 1, wherein the subject is human.

12. The method of claim 1, wherein the subject is an adult human.

13. The method of claim 1, wherein the subject is an adult, male human.

14. The method of claim 1, wherein the subject is a human child.

15. The method of claim 1, wherein the quantity of the PDE5A inhibitor is about 0.5 to 1.0 mg/kg body weight.

16. The method of claim 1, wherein the quantity of the PDE5A inhibitor is administered prior to a period of heavy exertion.

17. The method of claim 1, wherein the quantity of the PDE5A inhibitor is administered upon awakening.

18. The method of claim 1, wherein the quantity of the PDE5A inhibitor is administered prior to a planned period of physical exertion.

19. The method of claim 1, wherein the quantity of the PDE5A inhibitor is about 20 mg.

20. The method of claim 1, wherein the PDE5A inhibitor is tadalafil and the quantity is about 0.3 mg/kg body weight.

21. The method of claim 1, wherein the PDE5A inhibitor is tadalafil and the quantity is about 0.6 mg/kg body weight.

22. A method of treating Becker muscular dystrophy (BMD), comprising:
providing about 20 mg of tadalafil or a salt thereof; and
administering the about 20 mg of tadalafil or the salt thereof to an adult human subject in need of treatment for BMD.

23. The method of claim 22, wherein the adult human subject is male.

24. A method of treating Duchenne muscular dystrophy (DMD), comprising:
providing a quantity of tadalafil, sildenafil or a salt thereof; and
administering the quantity of tadalafil, sildenafil or a salt thereof to an human subject in need of treatment for DMD.

25. The method of claim 24, wherein the human subject is a child.

26. The method of claim 24, wherein tadalafil or a salt thereof is provided and the quantity is about 0.3 mg/kg body weight.

27. The method of claim 24, wherein the quantity of tadalafil, sildenafil or a salt thereof is about 0.5 mg/kg body weight.

28. The method of claim 24, wherein tadalafil or a salt thereof is provided and the quantity is about 0.6 mg/kg body weight.
